SEQUENCE LISTING (12 sequences)

SEQ ID NO: 1 - RNA, 22 nt, Artificial Sequence
  miRNA 146a
ugagaacugaauuccauggguu

SEQ ID NO: 2 - RNA, 22 nt, Artificial Sequence
  miRNA 21
uagcuuaucagacugauguuga

SEQ ID NO: 3 - RNA, 21 nt, Artificial Sequence
  siRNA Hsp70 SENSE
guuugucaguucucaauuuuu

SEQ ID NO: 4 - RNA, 21 nt, Artificial Sequence
  siRNA Hsp70 antisense
aaauugagaacugacaaacuu

SEQ ID NO: 5 - RNA, 21 nt, Artificial Sequence
  siRNA hSP70 #2 sense
ccaucuuacgacuauuucuuu

SEQ ID NO: 6 - RNA, 21 nt, Artificial Sequence
  siRNA hSP70 #2 antisense
agaaauagucguaagaugguu

SEQ ID NO: 7 - DNA, 19 bp, Artificial Sequence
  HSP70 siRNA 19-bp sequence
ggtggagatcatcgccaac

SEQ ID NO: 8 - DNA, 21 bp, Artificial Sequence
  siRNA GRP78 sense
gaaugaauuggaaagcuautt

SEQ ID NO: 9 - DNA, 21 bp, Artificial Sequence
  siRNA GRP78 antisense
auagcuuuccaauucauuctt

SEQ ID NO: 10 - RNA, 27 nt, Artificial Sequence
  siGRP78-1, sense
aauuucugccaugguucucacuaaaau

SEQ ID NO: 11 - RNA, 27 nt, Artificial Sequence
  siGRP78-2 sense
aacuucuacagcuucugauaaugaguc

SEQ ID NO: 12 - RNA, 20 nt, Artificial Sequence
  siRNA GRP78
agacgcuggaacuauugcuu

(19) United States
(12) Patent Application Publication
(10) Pub. No.: US 2022/0169709 A1
(43) Pub. Date: Jun. 2, 2022

(54) GRP78 AND/OR HSP70 INHIBITORS FOR THERAPEUTIC USE

(71) Applicant: GHP SCIENTIFIC LIMITED, London (GB)

(72) Inventor: Geltrude MINGRONE, Rome (IT)

(73) Assignee: GHP SCIENTIFIC LIMITED, London (GB)

(21) Appl. No.: 17/310,992

(22) PCT Filed: Mar. 4, 2020
(86) PCT No.: PCT/IB2020/051823

§ 371 Date: Sep. 3, 2021

(30) Foreign Application Priority Data
Mar. 6, 2019 (IT) 102019000003273

(51) Int. Cl. C07K 16/18; G01N 33/68; A61P 3/10; C12N 15/11

(52) U.S. Cl. CPC C07K 16/18; G01N 33/6893; A61K 2039/507; C12N 15/111; A61P 3/10

(57) ABSTRACT

The present invention relates to an association of inhibitors of the activity of at least one protein belonging to the Hsp70 family and of inhibitors of the GRP78 activity for use in the treatment of insulin resistance and/or of the pathologies deriving from this clinical condition.

The present invention relates to an association of inhibitors of the activity of at least one protein belonging to the Hsp70 family and of inhibitors of the GRP78 protein activity for use in the treatment of insulin resistance and/or of pathologies deriving from this clinical condition.

### STATE OF THE ART

It is known that insulin resistance is a pathological condition occurring when pancreatic islets secrete insulin but the hormone is unable to effectively trigger glucose metabolic uptake in metabolic tissue. The inability to absorb glucose by metabolic tissues results in hypoglycemia and hyperinsulinemia, which are symptoms of insulin resistance. Most individuals with insulin resistance have an often undiagnosed form, even though glycemic peak checking enables to highlight the pathology, and the absence of therapy can lead to the development of numerous pathologies for which insulin resistance constitutes a risk factor, such as obesity, cardiovascular disorders, hypertension, type-2 diabetes and other pathologies linked to this metabolic dysfunction.

For instance, the prevalence of obesity and type-2 diabetes (T2D) is rapidly increasing worldwide. OMS data demonstrate that, in 2014, 39% of men and 40% of women ≥18 y.o. in Europe were overweight, and that 10-30% of adults were obese. The 2011-2012 National Health and Nutrition Examination Survey in the U.S. demonstrated that 34.9% of ≥20 y.o. adults, age-adjusted value, were obese.

Obese subjects subjected to a hypocaloric diet rapidly lose weight during the first 1-2 weeks. However, a recent meta-analysis and review of the literature demonstrated that at +12 months from the start of lifestyle changes, addressed both to diet and physical activity, the average weight loss was only of −1.56 kg (95% confidence interval from −2.27 to −0.86 kg). This suggests that compliance to lifestyle changes is low and people should follow a hypocaloric diet throughout their lifespan, which occurs only rarely.

Bariatric surgery is very effective, both in causing a large weight loss and in maintaining lost weight. Moreover, bariatric surgery causes diabetes remission, or at least improves glycemic control, even long-term. However, only a small number, about 1%, of subjects eligible for bariatric surgery are actually offered surgery.

Duodenum bypass, obtained with Endobarrier or even acute infusion of nutrients through an endoscopically introduced nutrition probe which bypasses transit through the duodenum and a portion of the jejunum, significantly improves insulin sensitivity and reduces glycemia in T2D obese patients.

Duodenum and jejunum produce heat shock proteins (HSPs), that until some years ago were deemed to have only an intracellular protein transport function. Instead, in the last few years it was observed in the literature that HSPs can also be secreted and, therefore, are found in the bloodstream. However, their function is unclear and literature data are controversial.

Given the effects of insulin resistance, therapies capable of improving this pathological condition, with the entailed therapeutic/preventive effects for pathologies associated thereto or deriving therefrom, are of constant interest in medicine.

### SUMMARY OF THE INVENTION

The Authors of the present invention have discovered that obese subjects with T2D-associated or T2D non-associated insulin resistance exhibit high circulating (extracellular) levels both of Hsp70 and of GRP78 in response to a liquid high-fat, high-sucrose diet. However, when this liquid diet was injected through a nasal-jejunal tube downstream of the duodenum, this response was nearly totally abolished, leading to a significant improvement of insulin sensitivity and glycemic control. The Authors then studied the action of Sleeveballoon—a device by Keyron Ltd which is positioned endoscopically, reduces gastric volume and bypasses food transit into the duodenum—in insulin-resistant T2D rats, and discovered that heat shock proteins, in particular Hsp70 and GRP78, secreted by the duodenum during a high-fat diet, are accountable for insulin resistance and hyperglycemia, and verified in rat that the inhibition of their activity eliminates insulin resistance and improves glycemic control.

On the basis of the experiments and tests carried out, the Authors of the present invention discovered that the inhibition of the activity of Hsp70 protein and of GRP78 protein enables to treat insulin resistance and/or pathologies related thereto or deriving therefrom.

Therefore, object of the invention is the association of inhibitors of the Hsp70 protein activity or of the activity of a protein belonging to the HSP70 family and of inhibitors of the GRP78 activity for use in the treatment of insulin resistance and/or of pathologies deriving from or related to insulin resistance.

Moreover, the Authors of the present invention have further discovered that Hsp70 and GRP38 proteins can be used as biomarkers for the diagnosis of insulin resistance; therefore, object of the invention is a method for the diagnosis of insulin resistance in which the concentration of the two proteins is measured in blood samples.

A blood concentration of Hsp70 and GRP38 higher than 256 pg/ml is indicative of insulin resistance.

### Glossary

The inhibition can be at a protein, transcriptional, translational level.

By association in the present description it is meant an association of active ingredients both in the form of a physical mixture comprised of said active ingredients in a single dosage unit, and of dosage units physically separated by active ingredient (meant as inhibitor/s of Hsp70 or of a protein belonging to the HSP70 family and inhibitor/s of GRP78), but meant for a simultaneous or sequential administration such that each active ingredient provides the desired therapeutic effect substantially concomitantly with the other one.

According to the present description, by antibody fragments it is meant antibody fragments containing an antigen-binding site, binding an antigen of interest (in this case, specific HSP70 antigens, or antigens specific for a protein belonging to the HSP70 family as defined herein, or GRP78-specific antigens) such as Fab, Fv, dAbs fragments.

The term antibodies in the present description comprises monoclonal antibodies, recombinant antibodies and fusion proteins, in which an effector protein is bound to a fragment of antibody recognizing an HSP70-specific antigen, or recognizing an antigen specific for one of the proteins belonging to the HSP70 family, such as, e.g., those indicated in Table 0, or recognizing a GRP78-specific antigen.

### DETAILED DESCRIPTION OF THE FIGURES

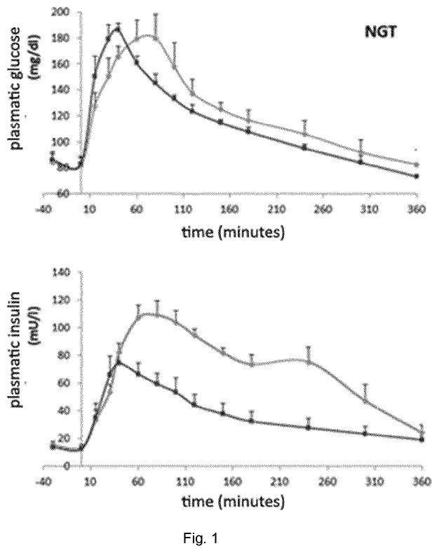

FIG. 1: Plasma glucose (upper panel) and plasma insulin (lower panel) curves after a liquid meal in NGT subjects. Grey rhombs and grey solid lines denote data after oral administration. Black squares and black lines denote data after meal administration into the jejunum.

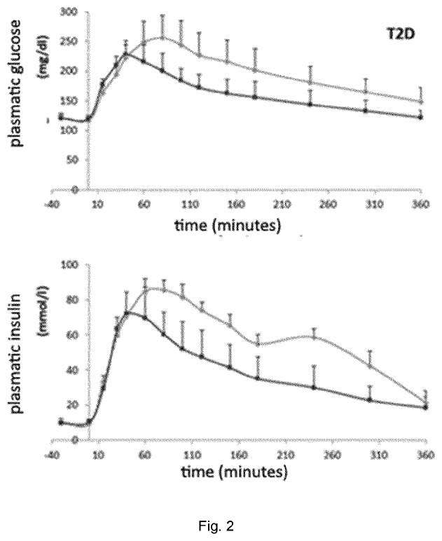

FIG. 2: Plasma glucose (upper panel) and plasma insulin (lower panel) curves after a liquid meal in T2D subjects. Grey rhombs and grey solid lines denote data after oral administration. Black squares and black lines denote data after meal administration into the jejunum.

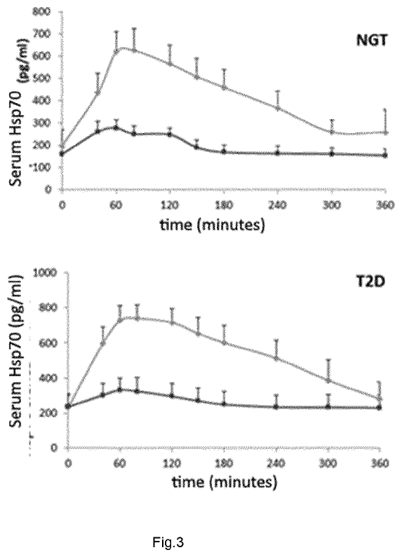

FIG. 3: Serum Hsp70 time curves after a liquid meal in NGT (upper panel) and T2D (lower panel) subjects. Grey rhombs and grey solid line denote oral administration of the liquid meal. Black squares and black solid line denote liquid meal injected directly into the jejunum.

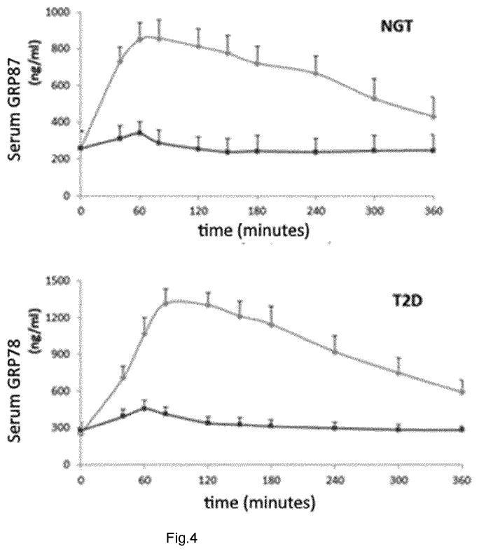

FIG. 4: Serum GRP78 time curves after a liquid meal in NGT (upper panel) and T2D (lower panel) subjects. Grey rhombs and grey solid line denote oral administration of the liquid meal. Black squares and black solid line denote liquid meal injected directly into the jejunum.

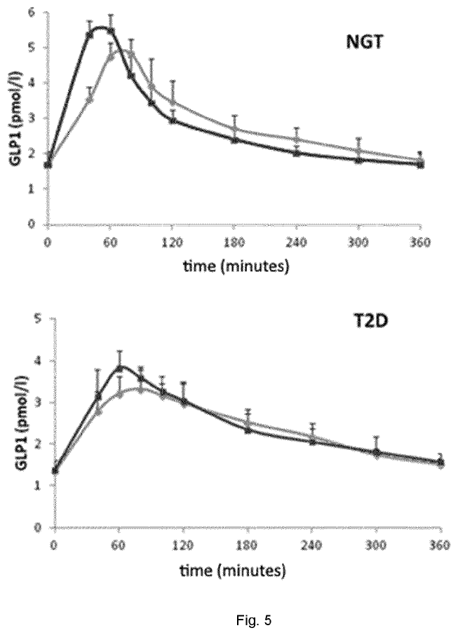

FIG. 5: GLP1 time curves after a liquid meal in NGT (upper panel) and T2D (lower panel) subjects. Grey rhombs and grey solid line denote oral administration of the liquid meal. Black squares and black solid line denote liquid meal injected directly into the jejunum.

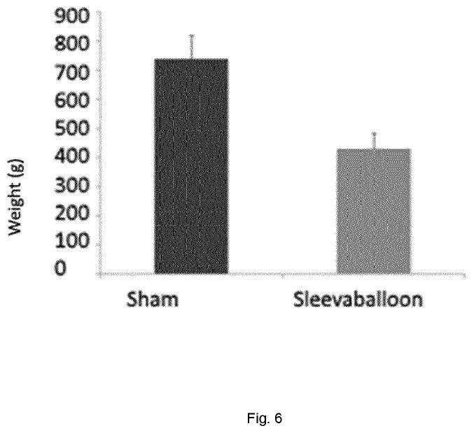

FIG. 6: Rat body weight at the end of the study (10 weeks of HFD before surgery and at +10 weeks after surgery).

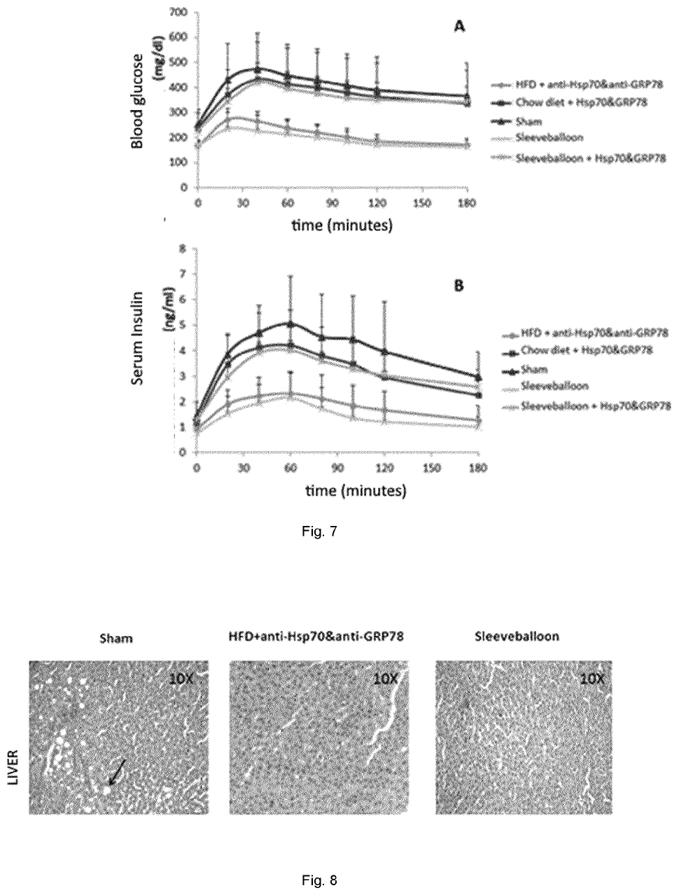

FIG. 7: Blood glucose (panel A) and serum insulin (panel B) levels after OGTT in sham rats, in rats implanted with a sleeveballoon, in rats implanted with a sleeveballoon and concomitantly with a continuous infusion pump infusing Hsp70+GRP78—all rats with HFD—and in rats with standard diet and continuous infusion of Hsp70+GRP78.

FIG. 8 haematoxylin/eosin stained liver sections.

The arrow indicates a lipid droplet.

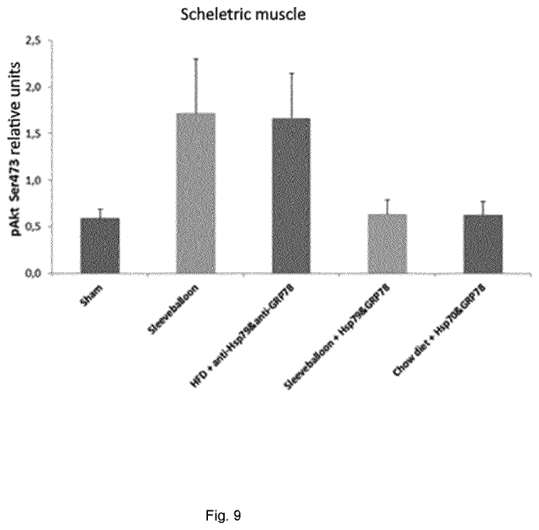

FIG. 9: Akt phosphorylated on Ser473, measured by multiplex assay in epitrochlear muscle biopsies.

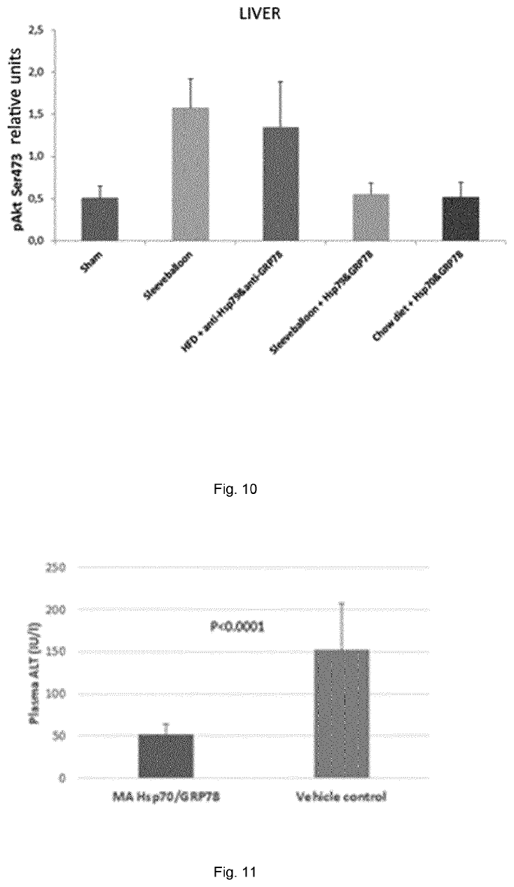

FIG. 10: Akt phosphorylated on Ser473, measured by multiplex assay in liver biopsies.

FIG. 11: FIG. 11 reports the alanine transaminase (ALT) plasma levels expressed as International Units per millilitre (IU/ml). In rats receiving antibodies against Hsp70/GRP78, ALT levels were one-third of the ALT concentrations in rats infused with vehicle control (52.33±11.86 vs. 152.33±55.01 IU/mi, P>0.0001).

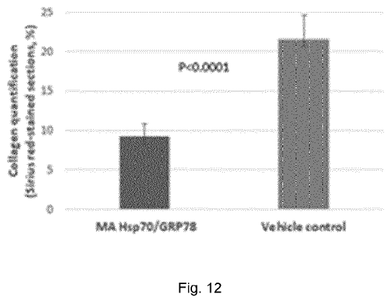

FIG. 12: FIG. 12 shows the percentages of Sirius red-stained collagen, which were 9.25±1.54 in rats infused with monoclonal antibodies against Hsp70/GRP78 and 21.58±3.03% in rats infused with vehicle control (P<0.0001).

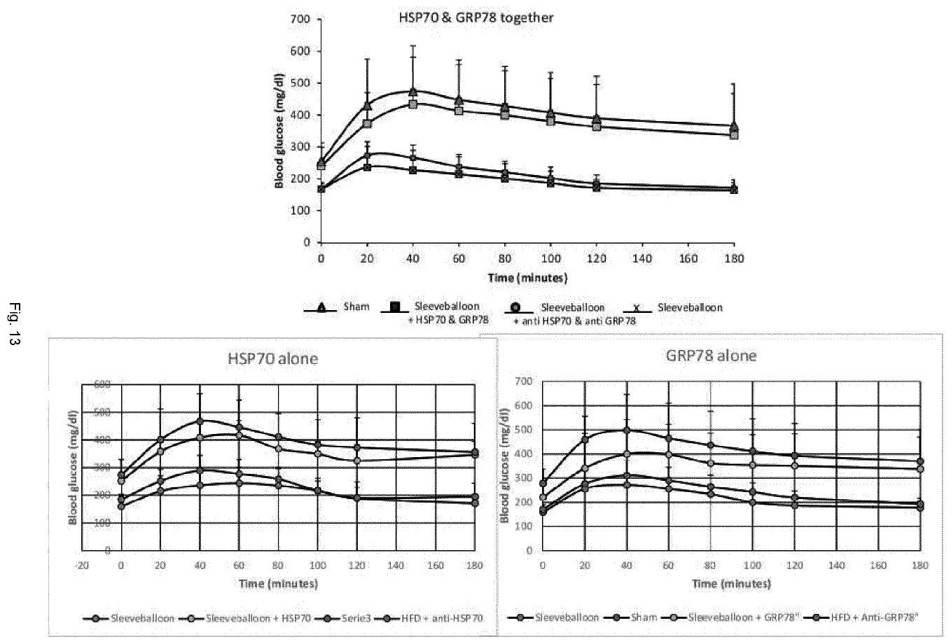

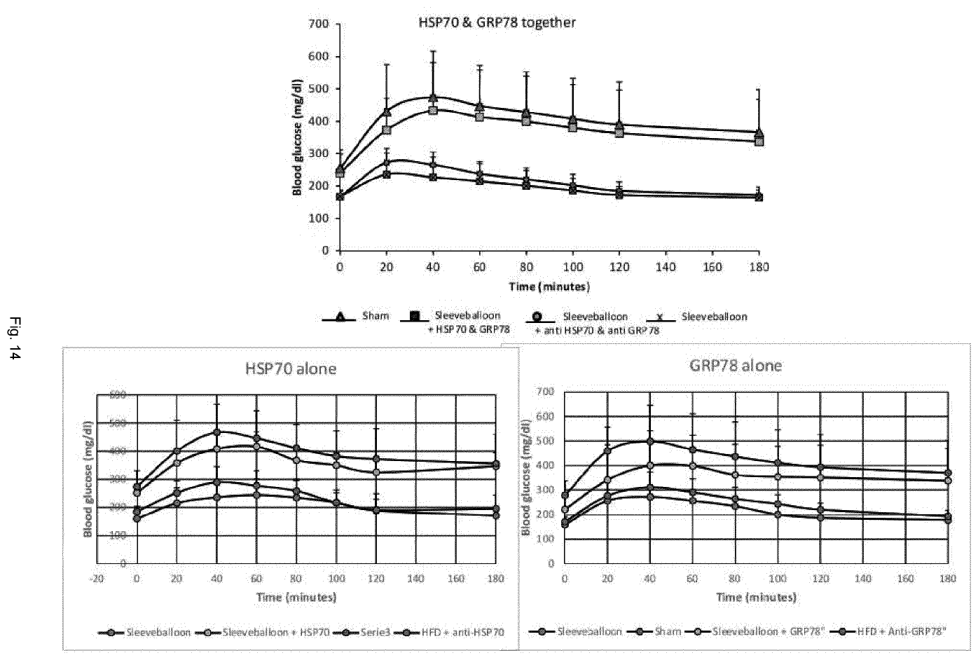

FIGS. 13 and 14 show the glycemia and insulinemia curves after OGTT in rats treated with high-fat diet (HFD) and sham-operated, HFD+Sleeveballoon, HFD+anti-HSP70 antibody and HFD+Sleeveballoon+HSP70 recombinant protein (rHSP70) vs. administration of both antibodies or recombinant proteins.

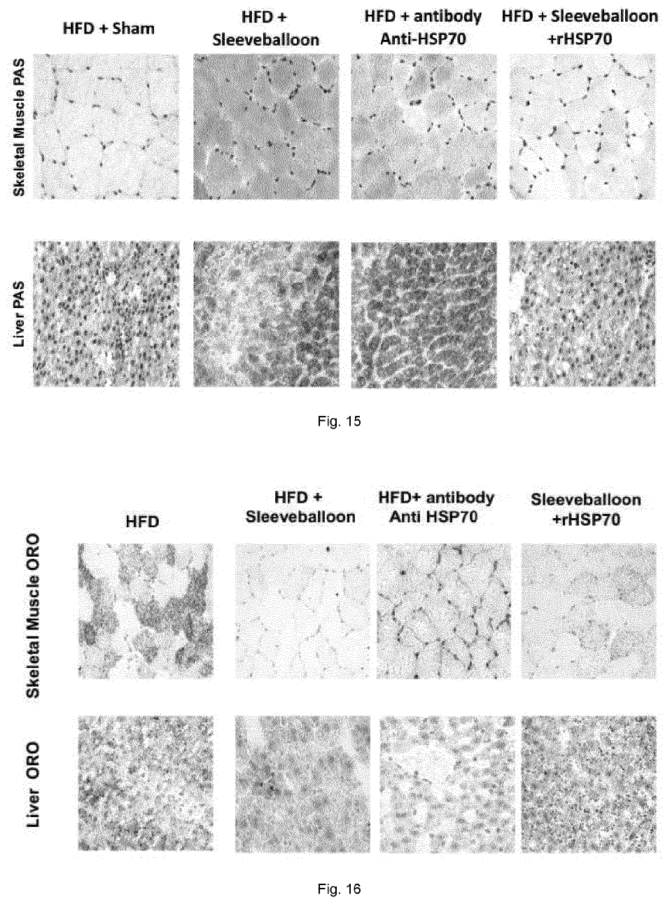

FIG. 15 shows the glycogen content, highlighted with PAS staining in the skeletal muscle and liver of rats treated with high-fat diet (HFD) and sham-operated, HFD+Sleeveballoon, HFD+anti-HSP70 antibody and HFD+Sleeveballoon+HSP70 recombinant protein (rHSP70).

FIG. 16 shows the lipid content (highlighted with ORO staining) in the skeletal muscle and liver of rats treated with high-fat diet (HFD) and sham-operated, HFD+Sleeveballoon, HFD+anti-HSP70 antibody and Sleeveballoon+HSP70 recombinant protein (rHSP70).

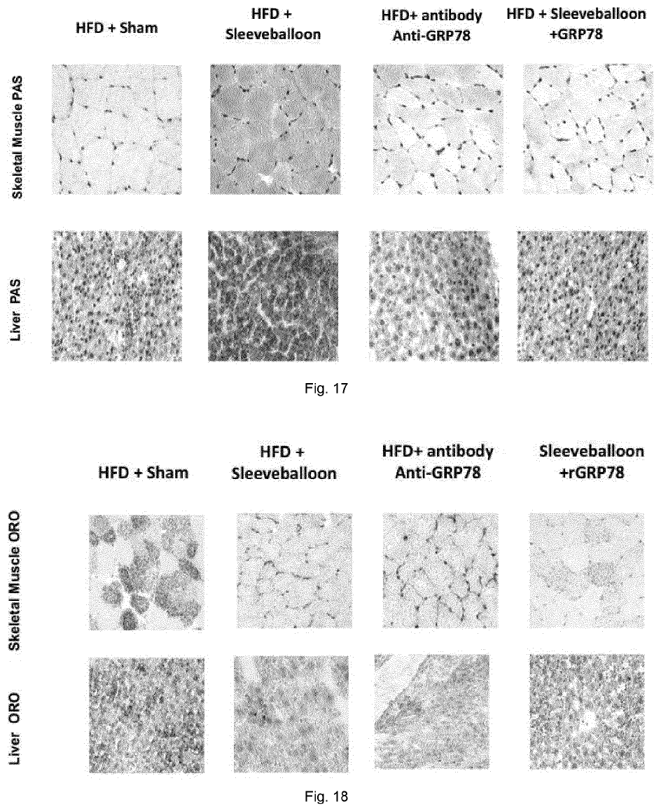

FIG. 17 shows the glycogen content, highlighted with PAS staining in the skeletal muscle and liver of rats treated with high-fat diet (HFD) and sham-operated, HFD+Sleeveballoon, HFD+anti-GRP78 antibody and HFD+Sleeveballoon+GRP78 recombinant protein (rGRP78).

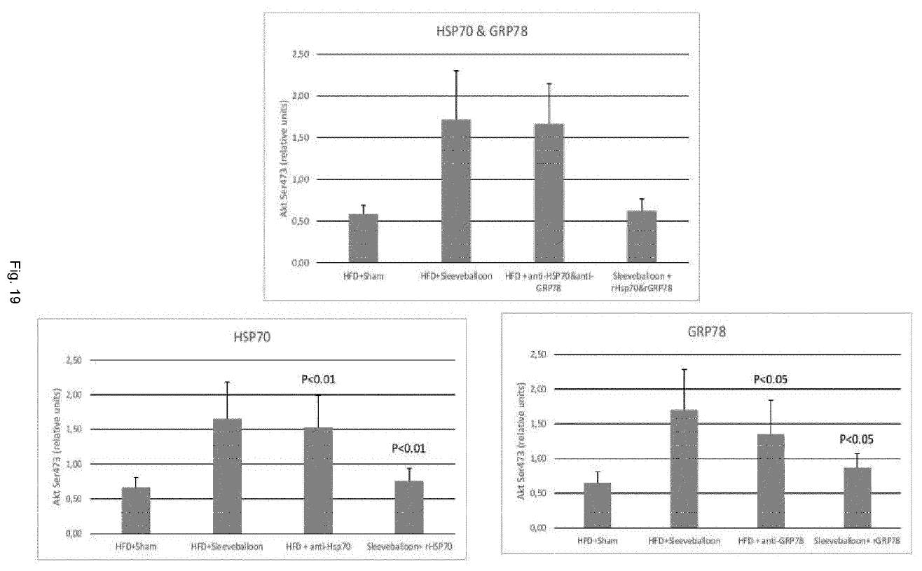

FIG. 18 shows the lipid content (highlighted with ORO staining) in the skeletal muscle and liver of rats treated with high-fat diet (HFD) and sham-operated, HFD+Sleeveballoon, HFD+anti-GRP78 antibody and Sleeveballoon+GRP78 recombinant protein (rGRP78).

FIG. 19 shows the effect of HSP70 or GRP78 administration, as recombinant proteins or as monoclonal antibodies, and shows a significant effect on insulin signalling.

SEQUENCE LISTING
SEQ ID NO 1:
ugagaacuga auuccauggg uu;
SEQ ID NO 2:
uagcuuauca gacugauguu ga;
SEQ ID NO 3
guuugucagu ucucaauuuu u;
SEQ ID NO 4:
aaauugagaa cugacaaacu u;
SEQ ID NO 5:
ccaucuuacg acuauuucuu u;
SEQ ID NO 6:
agaaauaguc guaagauggu u;
SEQ ID NO 7:
ggtggagatc atcgccaac;
SEQ ID NO 8:
gaaugaauug gaaagcuaut t;
SEQ ID NO 9:
auagcuuucc aauucauuct t;
SEQ ID NO 10:
aauuucugcc augguucuca cuaaaau;
SEQ ID NO 11
aacuucuaca gcuucugaua augaguc;
SEQ ID NO 12
agacgcugga acuauugcuu

### DETAILED DESCRIPTION

As reported in the examples of the present description, the Authors of the invention have discovered that the heat shock proteins Hsp70 and GRP78, secreted by the duodenum in response to a high-fat diet, are accountable for insulin resistance and diseases (pathologies) related thereto or triggered thereby.

The data provided in the examples and in the figures show a direct correlation between the secretion of said proteins in the duodenum and insulin resistance. Moreover, the data obtained on animals also show that an inhibition of the activity of said proteins on target tissues (skeletal muscle and liver), treats and/or prevents insulin resistance and prevents the development of diseases triggered thereby, such as diabetes.

Hence, object of the invention is an association of inhibitors of the activity of at least one protein belonging to the Hsp70 family and/or of inhibitors of the GRP78 activity for use in the treatment of insulin resistance and/or of pathologies deriving from or related to insulin resistance and as defined hereinafter.

The proteins belonging to the HSP70 family according to the present invention are those known in the literature, in particular those reported in Table 0 below.

| gene | protein | synonyms |
|---|---|---|
| HSPA1A | Hsp70 | HSP70-1, Hsp72 |
| HSPA1B | Hsp70 | HSP70-2 |
| HSPA1L | Hsp70 | |
| HSPA2 | Hsp70-2 | |
| HSPH2 | | HSPA4, HSP74 |
| HSPA5 | Hsp70-5 | BiP/Grp78 |
| HSPA6 | Hsp70-6 | HSP76 |
| HSPA7 | Hsp70-7 | |
| HSPA8 | Hsp70-8 | Hsc70, HSP71 |
| HSPA9 | Hsp70-9 | Grp75/mtHsp70 |
| HSPA12A | Hsp70-12a | |
| HSPA14 | Hsp70-14 | |

According to a particular embodiment, the proteins belonging to the HSP70 family are selected from HSP70, HSP71, HSP76, HSP74 and GRP75.

The inhibitors according to the invention can act directly on the activity of said proteins at an extracellular level, or can act at an intracellular level, e.g. by inhibiting the synthesis or the translation thereof, preferably in intestinal cells.

According to the present invention the inhibitors are inhibitors specific for each one of the proteins indicated, i.e., inhibitors specific for Hsp70 or for other proteins belonging to the HSP70 family as defined above and inhibitors specific for GRP78, i.e. inhibitors acting on the activity of the abovementioned proteins without interacting with other proteins. Examples of inhibitors according to the present invention are given by monoclonal antibodies specific for Hsp70 or for other proteins belonging to the HSP70 family as defined above or for GRP78, their fragments maintaining the specificity and the ability to inhibit Hsp70 activity, or the activity of other proteins belonging to the HSP70 family as above-defined, or of GRP78, miRNA, inhibiting the transcription of the mRNA encoding Hsp70 or other proteins belonging to the HSP70 family as above-defined or GRP78 or molecules inhibiting their receptors or blocking post-receptorial action.

A non-limiting example of inhibitors of the Hsp70 activity, or of the activity of other proteins belonging to the HSP70 family as defined above, is given by monoclonal antibodies or their fragments as defined above, specific for Hsp70 protein or for other proteins belonging to the HSP70 family as defined above (by the term specific it is meant binding uniquely the Hsp70 protein or a single protein belonging to the HSP70 family as defined above and inhibiting its activity), molecules of interfering RNAs such as siRNA that bind uniquely mRNA encoding Hsp70 or encoding a protein belonging to the HSP70 family as defined above.

According to the present description, it is deemed possible to use for the indicated therapeutic objects also toll-like receptor inhibitors TLR4 and/or TLR9, such as, e.g.: curcumin [(1E,6E)-1,7-bis-(4-hydroxy-methoxyphenyl)-1,6-heptadiene-3,5-dione] extracted from Curcuma longa, Sulforaphane (SFN) [1-isothiocyanate-4-(methylsulfinyl)butane] from cruciferae, iberin from cruciferae, Xanthoumol (XN) is a chalcone-type flavonoid from Humulus lupulus, celastrol pentacyclic triterpenoid isolated from Tripterygium wilfordii, berberine, atractylenolide I, zhankuic acid A, Rhodobacter sphaeroides lipid A (RsDPLA) which is a 1,4′-diphosphoryl penta-acyl lipid A, 1,4′-diphosphoryl penta-acyl lipid A from Bartonella quintana (BqLOS).

According to the present description, it is deemed possible to use for the therapeutic objects indicated, also synthetic toll-like receptor inhibitors TLR4 and/or TLR9 like, e.g.: Eritoran, miR-146a or micro RNA-146a (SEQ ID NO 1), miR-21 or micro RNA-21 (SEQ ID NO 2), NAHNP, HDL-like NP, Bare GNP, Glycolipid-coated GNP, Peptide-GNP hybrid.

According to the present invention, the monoclonal antibodies can be selected from any monoclonal antibody or fragment thereof capable of binding HSP70 and of inhibiting its activity, an example of such an antibody type can be given by HSP70 Monoclonal Antibody (3A3), or (5A5), or (4G4) or (MB-H1), or C92F3A Invitrogen Thermo Fisher Sci, or other anti-Hsp70 monoclonal antibodies available on the market. Monoclonal antibodies specifically binding individual proteins belonging to the HSP70 family are also available on the market, such as anti-GRP75 antibodies, monoclonal antibodies specific for HSP71, monoclonal antibodies specific for HSP76 and monoclonal antibodies specific for HSP74.

Molecules for interference on RNA (RNAi) can be selected from any siRNA capable of inhibiting the activity of the HSP70 protein or of a protein belonging to the HSP70 family as defined above, a non-limiting example of said siRNAs is given by one or more of (siRNA; Genolution, Seoul, South Korea) HSP70-1 (#1), SEQ ID NO 3 (Sense) and 5′ SEQ ID NO 4 (antisense), HSP70-1 (#2), SEQ ID NO 5 (Sense) and SEQ ID NO 6 (antisense) (Korea Human Gene Bank, Medical Genomics Research center, KRIBB, Korea).

HSP70 siRNA 19-bp SEQ ID NO 7, corresponding to nucleotides from 563 to 581 of HSP70 (GenBank accession # M11717).

A person skilled in the field will anyhow know how to design siRNAs capable of inhibiting protein HSP70 or proteins belonging to the HSP70 family as defined herein, by using appropriate programs available to the public, the nucleotide and amino acid sequences of said proteins, as well as the sequence of the mRNAs of said proteins, being known. Furthermore, the DNA-cutting protein Cas9 can be used, vehiculated in gastro-resistant capsules or in chitosan glutamate, which cuts DNA in the required zone, using a suitable guide RNA, thereby inhibiting protein synthesis in situ.

A non-limiting example of inhibitors of the GRP78 activity known and suitable for the carrying out of the present invention is given by monoclonal antibodies specific for the GRP78 protein (by the term specific it is meant that bind uniquely the GRP78 protein), molecules of interfering RNAs such as siRNAs that bind uniquely mRNA encoding GRP78.

The monoclonal antibodies can be selected among any monoclonal antibody or their fragment capable of binding the GRP78 protein and of inhibiting its activity, an example of said type of antibody can be given by GRP78 Monoclonal Antibody (C38), eBioscience™ directed towards the C terminus, or by GRP78 Monoclonal Antibody directed towards the N terminus.

Molecules for interference on RNA (RNAi) can be selected from any siRNA capable of inhibiting the activity of the GRP78 protein, a non-limiting example of such siRNA is given by GRP78 siRNA (sense SEQ ID NO 8 antisense SEQ ID NO 9) Sigma-Aldrich (St. Louis, Mo., U.S.A.) (mRNA, GenBank accession number NM_005347) or even siGRP78-1, sense: SEQ ID NO 10 and siGRP78-2, sense SEQ ID NO 10

Also for the inhibition of the activity of GRP78, vehiculated in gastro-resistant capsules or in chitosan glutamate, the DNA-cutting protein Cas9 can be used, which cuts DNA in the required zone, using a suitable guide RNA, thereby inhibiting protein synthesis in situ.

As indicated above, the inhibition can be performed by inhibiting the activity of one or more of the abovementioned proteins in circulating form, therefore extracellular, therefore by antibodies or their active fragments and/or by inhibiting, at intestinal level, the intracellular expression of one or both of the abovementioned proteins, therefore by molecules of interfering RNAs as above-defined.

In one particular embodiment the activity of one or both of the abovementioned proteins in extracellular form is inhibited, and also the activity of one or both of the abovementioned proteins at intracellular level in intestinal cells is inhibited.

Intracellular level inhibition localized in intestinal cells can be obtained by formulating molecules of interfering RNAs that bind uniquely mRNA encoding at least one protein belonging to the HSP70 family as defined herein and/or molecules of interfering RNAs that bind uniquely mRNA encoding GRP78 in gastro-resistant formulations which therefore enable the vehiculation of such inhibitors uniquely in the intestine, resulting in an intracellular inhibition circumscribed to intestinal cells.

Substantially, according to the present invention, the interfering RNA, like, e.g., the siRNA, will be used to silence the synthesis of at least one protein belonging to the HSP70 family as defined herein and/or of GRP78 exclusively in the intestinal mucosa, with preparations acting only at that level, oral administrations such as NPL, MPP or gastro-resistant capsules comprising the appropriate interfering RNAs, enabling the vehiculation of said RNAs in cells of the duodenum and of the jejunum. In fact, a diet high in fats and simple sugars stimulates the synthesis and secretion of these heat shock proteins that then, by coming into contact with membrane receptors of skeletal muscle and liver, determine insulin resistance and ectopic fat accumulation, besides glycogen content reduction. Insulin resistance determines a consequent insulin hyperproduction by the endocrine pancreas.

The monoclonal antibodies or their fragments as defined herein are instead directed against at least one protein of the HSP70 family as defined herein and/or circulating GRP78, i.e. present in blood, so as to block their effect, i.e., the induction of insulin resistance. Formulations enabling an intestine-localized intracellular inhibition by vehiculation of molecules of interfering RNAs to intestinal cells are known in the literature and will subsequently be described.

Any combination of one or more inhibitors of the activity of at least one protein belonging to the Hsp70 family and/or of one or more inhibitors of the GRP78 activity listed above is to be understood as covered by the scope of the present invention.

In one preferred embodiment, an association between one or more inhibitors of the activity of at least one protein belonging to the Hsp70 family, selected from the above-listed ones, and one or more inhibitors of the GRP78 activity selected from the above-listed ones is used.

Any combination between the two above-reported lists is an object of the invention.

In one embodiment, the association of the invention is represented by monoclonal antibodies, respectively specific against at least one protein belonging to the HSP70 family and GRP78 or silencing RNAs, respectively specific for Hsp70 and GRP78, e.g., selected from the above-described ones or a combination thereof. Alternatively, an association of monoclonal antibodies, specific against at least one protein of the Hsp70 family and of silencing RNAs specific for at least one nucleic acid encoding for a protein belonging to the HSP70 family, e.g. selected from the above-described ones or an association of monoclonal antibodies, specific against GRP78, and of silencing RNAs specific for RNA encoding GRP78, e.g., selected from the above-described ones, can be carried out. However, a person skilled in the art will know how to select or prepare other monoclonal antibodies or their fragments suitable for carrying out the invention or other siRNAs suitable for carrying out the invention, bearing in mind that said antibodies or their fragments or said siRNA must act so as to inhibit the activity of the respective proteins for which they are specific.

Object of the invention is also a therapeutic method for the treatment of insulin resistance and the treatment and/or the prevention of the diseases (pathologies) deriving therefrom or associated thereto in inhibitors, or an association thereof according to any one of the above-described embodiments is administered to a subject suffering from insulin resistance or from diseases deriving therefrom or associated thereto.

Anywhere in the present description and in the claims the term protein belonging to the HSP70 family can be replaced by any one of the proteins indicated in Table 0.

In one particular embodiment, the term protein belonging to the HSP70 family can be replaced, anywhere in the present description and in the claims, by the term HSP70, HSP71, HSP76, HSP74, and GRP75.

One example of diseases (pathologies) deriving from or associated to insulin resistance according to the present invention is represented by diabetes, hypertension, dyslipidemia, cardiovascular disease and other anomalies. These anomalies constitute insulin resistance syndrome. Since resistance usually develops well before these diseases appear, identifying and treating insulin-resistant patients has a potentially positive value. Insulin resistance can be, e.g., suspected in patients with a diabetes history in first-degree relatives; patients with a personal history of gestational diabetes, polycystic ovary syndrome or reduced glucose tolerance; and obese patients, in particular those with abdominal obesity.

Obesity, type 2 diabetes mellitus (previously known as non-insulin-dependent diabetes), hypertension, lipid disorders and heart diseases are common in most Western societies and are collectively accountable for an enormous burden of suffering. Many people have more than one—and sometimes all—of these conditions, leading to the hypothesis that the coexistence of these diseases is not a coincidence, but that an anomaly having a common basis allows them to develop. In 1988 the defect was suggested to be insulin-related, and insulin resistance syndrome was first described.

Insulin stimulates glucose absorption in tissues, and its ability to do so varies considerably among individuals. In insulin resistance, tissues exhibit a reduced response ability to insulin action. To compensate for the resistance, the pancreas secretes more insulin. Insulin-resistant individuals therefore have high plasma insulin levels. Among disorders appearing along with or developing in the wake of insulin resistance there are obesity, hypertension, dyslipidemia and type 2 diabetes, which are in fact associated with insulin resistance and compensatory hyperinsulinemia. It is known in the literature that the pathologies deriving from/triggered by or related with insulin resistance are, e.g., hyperglycemia, type 2 diabetes, NASH (non-alcoholic steatohepatitis), NAFLD (non-alcoholic fatty liver disease), metabolic syndrome, atherosclerosis, polycystic ovary syndrome, dyslipidemia, obesity, cardiovascular disease, hypertension.

In one embodiment, said diseases deriving from or associated with insulin resistance are selected from hyperglycemia, type 2 diabetes, metabolic syndrome, atherosclerosis, polycystic ovary syndrome, dyslipidemia, obesity, cardiovascular disease, hypertension, NASH, NAFLD and hepatic fibrosis. The data on rat reported in the experimental section show, e.g., the prevention of type 2 diabetes with inhibitors or their association according to the present description.

Object of the invention is also a composition comprising inhibitors of protein activity, of the activity of at least one protein belonging to the Hsp70 family and/or inhibitors of the GRP78 protein activity according to any one of the embodiments provided in the present description and one or more excipients or carriers pharmacologically acceptable for use in the treatment of insulin resistance and/or in the treatment or in the prevention of pathologies deriving from or related to insulin resistance.

According to one embodiment, therefore, the composition of the invention comprises one or more inhibitors of the activity of at least one protein belonging to the Hsp70 family that can be selected from one or more of the inhibitors listed above.

According to a further embodiment, the composition of the invention can therefore comprise one or more inhibitors of the GRP78 activity that can be selected from one or more of the inhibitors of GRP78 listed above.

The composition of the invention could comprise any combination of one or more inhibitors of the Hsp70 activity and of one or more inhibitors of the GRP78 activity listed above is to be understood as covered by the scope of the present invention.

All embodiments in which particular combinations of inhibitors for each of the two proteins are indicated for the association are to be understood as embodiments implementable also in the making of the composition of the invention.

Therefore, object of the invention is a composition comprising inhibitors of the activity of at least one protein belonging to the Hsp70 family and/or inhibitors of the GRP78 protein activity and one or more excipients or carriers pharmacologically acceptable for use in the treatment of insulin resistance and/or of pathologies deriving from or related to insulin resistance, wherein said inhibitors of at least one protein belonging to the HSP70 family are selected from one or more of monoclonal antibodies or their specific fragments for Hsp70 protein or for a protein belonging to the HPS70 family as defined herein and/or molecules of interfering RNAs that bind uniquely mRNA encoding Hsp70 or a protein belonging to the HPS70 family and said inhibitors of the GRP78 activity are selected from one or more of monoclonal antibodies or their specific fragments for the GRP78 protein and/or molecules of interfering RNAs that bind uniquely mRNA encoding GRP78.

In one embodiment, the composition could e.g. comprise one or more monoclonal antibodies or their specific fragments for Hsp70 protein or for a protein belonging to the HSP70 family and/or one or more monoclonal antibodies or their specific fragments for the GRP78 protein.

In case the composition comprises one or more monoclonal antibodies or their specific fragments for Hsp70 protein or for a protein belonging to the HSP70 family and monoclonal antibodies or their specific fragments for the GRP78 protein, said antibodies or their fragments could be comprised in a single formulation or could be in separate formulations to be administered simultaneously or sequentially.

In another embodiment the composition may comprise interfering RNA molecules specific for mRNA encoding Hsp70 or a protein belonging to the HSP70 family and/or specific for mRNA encoding GRP78.

Said molecules of interfering RNAs could be siRNAs, e.g. siRNAs as defined above, e.g. siRNAs selected from one or more of SEQ ID 1 to 11.

In case the composition comprises one or more molecules of interfering RNAs as defined above, those could be comprised in a single formulation or could be in separate formulations to be administered simultaneously or sequentially.

Since in case of interfering RNA molecules (therefore exerting inhibition at intracellular level) an inhibition of intestinal cells is desired, said molecules will preferably be formulated in a form for oral use, gastro-resistant, like capsules, gel, softgel or other appropriate forms for the vehiculation and the release of drugs at the intestinal level.

A particular embodiment of the present invention is represented by a pharmaceutical composition in the form of association for simultaneous or sequential administration, in which the different classes of active ingredients, i.e., antibodies and molecules of interfering RNAs, are respectively provided in formulations for subcutaneous administration and in gastro-resistant formulations for oral administration.

As to the preparing of gastro-resistant formulations, the technician in the field knows that there are more than 10 US Food and Drug Administration (FDA) approved pharmaceuticals employing lipid nanoparticle (LNP) as drug delivery systems.

To carry out the invention, LNP systems loaded with one or more siRNAs as described herein, containing a ionizable lipid, optionally comprised in gastro-resistant formulations, can be used.

The technician in the field could select any one of the systems known in the literature for the oral administration of molecules of interfering RNAs, preferably selecting systems already validated for the vehiculation of said molecules at the intestinal level.

One example of ionizable lipid is represented by 1,2-dilinoleyl-N,Ndimethyl-3-aminopropane (DLinDMA),

Alternatively, LNP systems loaded with one or more siRNAs as described herein can be created, containing ionizable cationic lipids that possess an amine function and an acid dissociation constant (pKa) of about 6.5 (3).

An example of said lipids is represented by the ionizable lipid form DLin-KC2-DMA described in detail in the materials and methods and in the article Semple, S. C. et al. Rational design of cationic lipids for siRNA delivery. Nat. Biotechnol. 2010; 28: 172-176.

In one embodiment, the LNP loaded with the siRNAs described herein can be directly administered orally, without being embedded in a gastro-resistant capsule, at fasting, whereby they come into contact with very small concentrations of pepsin and bile acids and do not encounter conformational changes, enabling the interiorization of siRNA-containing LNP into the intestinal mucosa.

Alternatively, it is possible to use mucus penetrating nanoparticles, known to the technician in the field as MPPs (mucus penetrating particles) which diffuse through mucus at speeds comparable to diffusion in water. It is known that MPPs are ˜100 nm in diameter, neutrally charged, and have high levels of low molecular weight polyethylene glycol (PEG) on their surface.

MMPs characterized by being coated of PEG are known.

Alternatively, the interfering RNAs could be administered in gastro-resistant capsules opening into the duodenum or the jejunum, prepared by any technique known in the field.

The dose of siRNA (HSP70, or at least a protein belonging to the HSP70 family and/or GRP78 as described herein) that might be administered in humans can vary from 0.01 to 3 mg/kg of weight.

siRNA formulations that can be used in humans are:

To silence the intestinal synthesis of HSP70, or of at least a protein belonging to the HSP70 family, MISSION HSPA1A siRNA (SASI_Hs01_00051449; Sigma-Aldrich, St. Louis, Mo., USA) can be used at a final concentration of 50 nM in cationic liposome (like, e.g., Lipofectamine RNAiMAX Reagent; Invitrogen, Carlsbad, Calif., USA) or one of the siRNAs described herein and provided in SEQUENCE LISTING.

To silence the intestinal synthesis of GRP78, siRNAs having SEQ ID NO 12 (Genechem Corporation (Shanghai, China)) vehiculated in lipofectamine 2000 (Invitrogen) can be used.

The composition of the invention could be used for the treatment of insulin resistance and/or for the treatment or the prevention of pathologies related thereto or deriving therefrom. Said pathologies are, e.g., hyperglycemia, type 2 diabetes, metabolic syndrome, atherosclerosis, polycystic ovary syndrome, dyslipidemia, obesity, cardiovascular disease, hypertension, NASH, NAFLD and hepatic fibrosis. Therefore, object of the invention is also a method for the treatment of insulin resistance and/or for the treatment or the prevention of pathologies related thereto or deriving therefrom, represented, e.g., by the above-listed ones, comprising the administration of the composition of the invention to a subject in need thereof.

The composition of the invention could be made in a formulation suitable for oral, systemic, intranasal or intravenous administration.

Preferably, in case of administration of antibodies a formulation for systemic-type, in particular subcutaneous administration will be preferred.

In case of administration of molecules of interfering RNAs, a gastro-resistant formulation for oral administration will be preferred, like, e.g., NLPs or MPPs as above-described.

Both in the association and in the composition according to the invention, the inhibitors, when in the form of monoclonal antibodies, can be provided for each target protein in the amount of 50-250 ng/kg, e.g., by subcutaneous injection. The administration of each dosage of antibody could be simultaneous or sequential, and could be repeated once a week or every 2 weeks. In one embodiment, each antibody could be administered, in combination or sequentially, in a formulation causing their delayed release, e.g. by linking to the antibody albumin and/or fatty acids and/or dicarboxylic acids.

Moreover, object of the invention is a method for the diagnosis of insulin resistance in a human being in which:
a. measuring the concentration of a blood sample of Hsp70 and GRP78 proteins
b. a blood concentration of said proteins higher than 256 pg/ml is indicative of insulin resistance.

The blood sample may be a sample of whole blood or of plasma or serum.

According to the invention, it is also possible to use the diagnostic method of the invention within the scope of a therapeutic treatment as described herein, in which the concentration of Hps70 and GRP78 proteins is verified as initial step, to then proceed with the administration of the inhibitors as defined herein and according to the description.

Anywhere in the description and in the claims the terms HSP70, HSP70 family and GRP78 can be replaced respectively by the terms human HSP70, human HSP70 family, human GRP78.

In the following examples the experimental data obtained by the Inventors are provided, demonstrating the direct correlation between Hsp70 and GRP78 expression in the duodenum and insulin resistance pathology.

All experiments on humans were carried out with the approval of the Ethics Committee of the Università Cattolica del Sacro Cuore of Rome, Italy, and the subjects enrolled signed an informed consent to the study. All studies on animals were approved by the Ethics Committee for Animal Experimentation of the Università Cattolica del Sacro Cuore of Rome, Italy.

### Examples

1. Human Studies

Subjects

Six insulin-resistant subjects, of which 3 with and 3 without T2D, were enrolled in the study after said study was approved by the Ethics Committee of the Università Cattolica of Rome, Italy. All subjects signed the informed consent to the study according to the ethical principles of the Declaration of Helsinki regarding human experimentation.

The inclusion criteria were: men and women aged 30-60 years, a body mass index (BMI) of 30-40 kg/m², a glycated hemoglobin (HbA_{1c}) of from 6,5% to 8.5% (for diabetic patients).

Subjects with normal glucose tolerance (NGT) were recruited on the basis of the absence of diabetes after an oral glucose load curve. All diabetic subjects were newly-diagnosed and insulin-resistant (HOMA-IR >2.60).

Exclusion criteria were the presence in active state of major diseases of endocrinological, renal, cardiac, respiratory, hepatic or gastrointestinal type.

The patients randomly received a liquid meal, administered orally or into the jejunum.

In one session, the subjects received Calogen Extra (Nutricia) 200 ml (400 kcal/100 ml)—containing 4 g/100 ml of proteins, 4.5 g/100 ml of carbohydrates and 40.3 g/100 ml of fats—with the addition of 40 g of sucrose (157.6 kcal) so as to obtain the following meal composition: proteins 8 g, simple carbohydrates 49 g and fats 80.6 g.

In another session the intestine was incannulated by means of a 240 cm-long 10 French probe (Cook Medical, Bloomington, Ind.). the tube was connected to a balloon occluding the intestinal lumen upstream of the infusion site. The same liquid meal was injected into the jejunum in less than 10 minutes.

Blood samples were collected at −30 min and at 0, 15, 30, 40, 60, 80, 100, 120, 150, 180, 240, 300, and 360 min to dose glucose, insulin and glucagon-like peptide-1 (GLP-1) during each study (some points less for GLP1).

Analysis Methods

Glycemia was measured with Analox GM9 Glucose Analyzer (Beckman Instruments, Fullerton, Calif.) immediately after collection.

Plasma insulin was measured by ARCHITECT® chemiluminescent immunoassay (Abbott Laboratories).

GLP-1 (7-36)amide/(7-37) was measured with GLP-1 (active) enzyme-linked immunoassay kit (Linco). This dosage is based on a monoclonal antibody coating the wells and binding the NH₂ terminus region of the active GLP1. GLP1 concentration is proportional to the fluorescence generated by umbelliferone, which is produced by the reaction between alkaline phosphatase bonded to anti-GLP1 monoclonal antibodies and methyl-umbellipheryl-phosphate. The method detection limit is 2 pmol/l; the variation coefficient inside a single procedure (VC) is 8%, both at low and high concentrations (range 4-76 pmol/l), and between different procedures the VC is 12% for 4-8 pmol/l and 7% for 28-76 pmol/l. Cross-reactivity is 100% for GLP-1 (7-36)amide and GLP-1 (7-37), but is not measurable for GLP-1 (9-36)amide, GLP-2, and glucagone.

Statistics

All data are expressed as means±SD, unless specified otherwise. Wilcoxon test was used to compare data inside the same group. P<0.05 is deemed significant.

Insulin sensitivity during meals was measured by Oral Glucose Insulin Sensitivity (OGIS) model (13). OGIS provides an insulin sensitivity index which is analogous to that obtained by the clamp.

2. Animal Studies

Animals

Rats were stabulated at the Stabularium of the Universitá Cattolica of Rome (Italy) under standard and controlled conditions, with free access to food and water. AH procedures on animals were approved by the Ethics Committee for Animal Experimentation of the Universitá Cattolica del Sacro Cuore of Rome, Italy.

The study consisted of 5 groups of animals. Wistar rats (250-300 g) were fed ad libitum with a high-fat diet (HFD) containing purified tripalmitin (Rieper AG, Bolzano, Italy) which provides 71% of energy from saturated fats, yet corn oil (1.9/100 g diet) was also present so as to prevent essential fatty acid deficits, 20% of carbohydrates including corn starch and sucrose (2:1 weight/weight) and 10% proteins, for 10 weeks under controlled conditions (12:12 hours of light/dark cycles, 50-60% humidity, 25° C. with free access to food and water apart when prescribed) prior to sleeveballoon positioning or sham surgery. Rats received HFD for 10 weeks post-surgery and surgeons were always the same ones.

Group A consisted of rats subjected to sham surgery (n=7). Group B consisted of HFD rats implanted with a sleeveballoon (n=7). Group C consisted of 7 rats with a standard diet, in which a rechargeable infusion pump (Alzet osmotic pump, Cupertino, Calif.) was subcutaneously implanted, said pump containing both Hsp70 and GRP78 with an infusion rate of 500 pg/hour for each heat shock protein (total 1000 pg/ml) which were infused for 10 weeks. Group D consisted in 7 rats with HFD in which an infusion pump was positioned under the skin of the cervical region, continuously infusing monoclonal antibodies (HSP70 Monoclonal Antibody (5A5), Thermo Fisher Scientific; GRP 78 Antibody (76-E6), Santa Cruz Biotechnology) against Hsp70 and GRP78 with a total infusion rate of 1000 pg/hour (500 pg for Hsp70 and 500 pg for GRP78) for 10 weeks. Group E, in which 7 rats were implanted with the sleeveballoon and concomitantly with an infusion pump positioned under the skin of the cervical region and continuously infusing Hsp70 and GRP78 with an infusion rate of 500 pmol/hour each, for a total of 1000 pmol/hour for 10 weeks.

Surgery

The rats were anesthetized with ketamine (75 mg/kg intramuscular) and xylazine (10 mg/kg intramuscular). 10 ml of 0.9% NaCl in sterile solution were administered subcutaneously prior to surgery.

A 3 cm laparotomy incision was performed on the abdominal wall, and the stomach was isolated outside of the abdominal cavity and laid on gauzes soaked with saline. Both in sham-operated rats and in the sleeveballoon group a small cut was made into the wall of the gastric bottom to introduce the device (Sleeveballoon made of silicon with a central channel and continuing with a 10 cm, 10-12 French catheter) or for sham surgery.

The rats, stabulated in individual cages, were weighed weekly and studied at +10 weeks from surgery.

The cut on the gastric wall was then sutured and the stomach repositioned in the abdominal cavity. Finally, the abdomen wall was closed in layers. After surgery, the rats received for 3 days 10 ml/day of saline under skin and 0.3 ml of Buprenex. Prior to surgery, rats were left fasting for 24 hours, and after surgery for 3 days had access to an Osmolite OneCal liquid diet, until when they were again fed normally.

Post-Surgical Studies

A basal collection was obtained after 6-8 hours of fasting. Oral glucose tolerance tests (OGTT; 1 g/kg of body weight) curve were administered with gastric gavage. Blood samples were obtained at 0, 20, 40, 60, 80, 100, 120 and 180 minutes after glucose bolus to measure glycemia and insulinemia. At the end of the OGTT, blood was collected by cardiac puncture and put into test tubes containing EDTA, aprotinin and dipeptidyl peptidase 4 (DPP-4) inhibitor and the glucagon-like peptide 1 (GLP1) analyzed. After centrifuging, the plasma was split into aliquots and subsamples and stored at −20° C. until analysis.

Histology

Rats were sacrificed and fresh liver sections were taken for each rat, fixed in (10%) formalin buffer and dehydrated with ethanol gradients (70%, 80%, 90%, 95% and 100%). After dehydration, the samples were put in xylene. Finally, the samples were embedded in paraffin and sliced at the microtome (3-4 μm). The slices were stained with haematoxylin-eosin. The sections were photographed with an optical microscope (ZEISS Primo Star HAL/LED).

Analysis of Tissue Phosphoproteins

Biopsies of epitrochlear muscles and of liver were minced and put in dry ice with Tissue Protein Extraction Reagent (Thermo Scientific, Rockford, Ill., USA) with the addition of a protease cocktail and of a phosphatase inhibitor (Thermo Scientific). The very small pieces of tissue were then homogenized with a TissueRuptor (Qiagen) and stirred with a vortex for 10 min at 4° C. Samples were then centrifuged at 12000 g for 10 min and the supernatant collected and stored at −80° C. until analysis. Total protein content was measured with BCA protein assay (Thermo Scientific).

Then, a kit for total protein and phosphoprotein dosage, with magnetic beads for Akt^{Ser473}, GSK3α^{Ser21}, GSK3β^{Ser9}, IGF1R^{Tyr1131}, IRS1^{Ser636/639}, ERK1/2^{Thy202/204, Thy185/187} was used.

Mean fluorescence intensity was measured for each analyte with Bio-Plex 200 System (Bio-Rad, Hercules, Calif., USA). Mean fluorescence intensity of a particular phosphorylated protein was normalized for total protein amount to obtain the related units.

For the sake of simplicity, the Inventors report only the Akt data.

Analysis Methods

Blood glucose levels were analyzed with Accu-Check Mobile glucometer (Roche Diabetes Care).

Serum insulin was measured by an ELISA kit ultrasensitive for rat insulin (BioVendor, Kassel, Germany), with a sensitivity of 0.025 ng/mL and a 10% accuracy within the dosage and among various dosages. GLP-1₇₋₃₆ in plasma was measured with a rat-specific ELISA for rodents (Millipore, St. Charles, Mo.). Sensitivity for GLP-1₇₋₃₆ was of 5.2 pg/mL, <11% variability in the same dosage, <19% variability for different dosages, and 83% accuracy.

Statistical Analysis

The HOMA index for insulin resistance (HOMA-IR) (13) was calculated as glycemia at fasting (mg/dL)×plasma insulin (μU/mL)/405. Factor 405 takes into account the measuring units.

The area under curve (AUCs) was calculated by trapezoidal method. Post-OGTT insulin sensitivity as glucose AUC/insulin AUC ratio. Differences between groups were calculated by Mann-Whitney U after having tested the lack of normal distribution by Shapiro-Wilk test.

Differences were considered significant for P<0.05. Spearman correlation was used to verify correlations among variables.

FIG. 7 clearly shows that HSP70 and GRP78 administration causes insulin resistance and hyperglycemia, whereas the administration of the respective monoclonal antibodies causes a significant improvement of insulin sensitivity and a remarkable reduction of glycemic values.

Assessment of Insulin Sensitivity and of Hsp70 and GRP78 Proteins in Blood Samples.

HSP70 and GRP78 were validated as biomarkers in another cohort of 23 subjects (12 women and 11 men) with a BMI of 35.75±7.41 kg/m² (from 24.62 to 46.51) and with a mean age of 44.09±13.23 years. Insulin sensitivity was assessed by Oral Glucose Insulin Sensitivity (OGIS) model (Mari, G. Pacini, E. Murphy, B. Ludvik and J. J. Nolan. A Model-Based Method for Assessing Insulin Sensitivity from the Oral Glucose Tolerance Test. Diabetes Care 24:539-548, 2001). Mean OGIS value was 337.17±66.11 ml/min/m² and mean level of HSP70 and GRP78 was of 285.70±141.47 pg/ml.

The area under curve (AUC) of the receiver operating characteristics (ROC) was of 0.95±0.04 P<0.0001. The optimal cut-point was of 256 pg/ml, a value beyond which the subjects were insulin-resistant and the optimum criteria 0.83, according to Youden's method.

Results

Study on Humans

Subjects with NGT or T2D were paired by gender (2 males and 1 female in NGT and 2 males and 1 female in diabetic subjects), by age (48.67±4.72 years in NGT and 50.67±2.08 years in diabetic subjects), and BMI (43.60±0.93 kg/m² in NGT subjects e 42.41±0.94 kg/m² in diabetic subjects). In T2D subjects, the mean value of HbA_{1c} was 7.75%, this means that subjects were in a state of moderate diabetic decompensation.

Glucose and insulin AUCs were significantly higher when the liquid meal was given orally rather than injected directly into the jejunum bypassing the duodenum, and this both in NGT subjects (FIG. 1) and in T2D ones (FIG. 2).

The glucose AUC/insulin AUC ratio was significantly higher when the meal was directly administered into the jejunum; this means that the amount of glucose eliminated from the bloodstream per insulin unit was higher. Insulin sensitivity measured with OGIS was also significantly higher when the meal bypassed the duodenum (Table 1).

TABLE 1

| | | P (Wilcoxon |
|---|---|---|
| | Mean ± | signed rank |
| | SD | test) |
| oral glucose AUC | 56568.33 ± | 0.028 |
| | 15949.22 | |
| fasting glucose AUC | 48822.92 ± | |
| | 10393.43 | |
| oral insulin AUC | 23339.77 ± | 0.028 |
| | 3928.66 | |
| fasting insulin AUC | 12641.04 | |
| | 2367.88 | |
| oral glucose/insulin ratio | 2.41 ± | 0.028 |
| AUC | 0.45 | |
| fasting glucose/insulin ratio | 3.90 ± | |
| AUC | 0.67 | |
| Oral OGIS | 296.38 ± | 0.028 |
| | 54.20 | |
| fasting OGIS | 351.43 ± | |
| | 74.01 | |
| oral HSP70 AUC | 164081.67 | 0.028 |
| | 35322.63 | |
| fasting HSP70 AUC | 77669.17 ± | |
| | 20802.20 | |
| oral GRP78 AUC | 281156.67 ± | 0.028 |
| | 65622.55 | |
| fasting GRP78 AUC | 93714.17 ± | |
| | 20141.69 | |
| oral GLP1 AUC | 1057.03 ± | 0.345 |
| | 180.73 | |
| fasting GLP1 AUC | 1080.20 ± | |
| | 158.50 | |

Area Under Curve (AUC) of glucose and insulin levels; glucose AUC/insulin AUC ratio; insulin sensitivity measured with OGIS; Hsp70 AUC, GRP78 AUC and GLP1 levels AUC after liquid meal. All measurements relate to the two administration modes (oral and jejunal).

The curve of Hsp70 circulating levels after the liquid meal orally or intrajejunally administered in NGT or T2D subjects is reported in FIG. 3, whereas that of GRP78 is reported in FIG. 4.

The GLP1 time curve both in NGT subjects and in T2D ones is reported in FIG. 5 and shows that the peak is anticipated when the meal is administered into the jejunum, but without significant AUC increase (Table 1).

A multiple regression with OGIS as dependent variable and Hsp70 AUC, GRP78 AUC and GLP1 AUC as independent variables demonstrated that the R² of the model was 0.772 with P<0,0001: the partial correlation with GRP78 AUC was −0.879 (P<0.0001), the partial correlation with Hsp70 AUC was −0.235 (P=0.043), whereas the partial correlation with GLP1 AUC was 0.098, P=0.487. Therefore, GRP78 AUC was the variable explaining most of the variation of OGIS insulin sensitivity.

Hsp70 and GRP78 serum concentrations, as demonstrated by their AUCs (Table 1), were drastically reduced when the liquid meal was administered into the jejunum.

The correlations among glucose AUC, insulin AUC, OGIS, Hsp70 and GRP78 are reported in Table 2 hereinafter.

TABLE 2
Correlations according to Spearman.

| | | glucose | Insulin | G/I AUC | | Hsp70 | GRP78 | GLP1 |
|---|---|---|---|---|---|---|---|---|
| | | AUC | AUC | ratio | OGIS | AUC | AUC | AUC |
| Glucose | Correlation | 1.000 | .622(*) | .182 | −.986(**) | .874(**) | .937(**) | −.587(*) |
| AUC | coefficient | | | | | | | |
| | Sig. (2-tailed) | . | .031 | .572 | .000 | .000 | .000 | .045 |
| | N | 12 | 12 | 12 | 12 | 12 | 12 | 12 |
| insulin AUC | Correlation | .622(*) | 1.000 | −.622(*) | −.650(*) | .720(**) | .706(*) | −.245 |
| | coefficient | | | | | | | |
| | Sig. (2-tailed) | .031 | . | .031 | .022 | .008 | .010 | .443 |
| | N | 12 | 12 | 12 | 12 | 12 | 12 | 12 |
| G/I AUC | Correlation | .182 | −.622(*) | 1.000 | −.140 | −.028 | .035 | −.217 |
| Ratio | coefficient | | | | | | | |
| | Sig. (2-tailed) | .572 | .031 | . | .665 | .931 | .914 | .499 |
| | N | 12 | 12 | 12 | 12 | 12 | 12 | 12 |
| OGIS | Correlation | −.986(**) | −.650(*) | −.140 | 1.000 | −.888(**) | −.944(**) | .636(*) |
| | coefficient | | | | | | | |
| | Sig. (2-tailed) | .000 | .022 | .665 | . | .000 | .000 | .026 |
| | N | 12 | 12 | 12 | 12 | 12 | 12 | 12 |
| AUC Hsp70 | Correlation | .874(**) | .720(**) | −.028 | −.888(**) | 1.000 | .930(**) | −.566 |
| | coefficient | | | | | | | |
| | Sig. (2-tailed) | .000 | .008 | .931 | .000 | . | .000 | .055 |
| | N | 12 | 12 | 12 | 12 | 12 | 12 | 12 |
| AUC GRP78 | Correlation | .937(**) | .706(*) | .035 | −.944(**) | .930(**) | 1.000 | −.566 |
| | coefficient | | | | | | | |
| | Sig. (2-tailed) | .000 | .010 | .914 | .000 | .000 | . | .055 |
| | N | 12 | 12 | 12 | 12 | 12 | 12 | 12 |
| AUC GLP1 | Correlation | −.587(*) | −.245 | −.217 | .636(*) | −.566 | −.566 | 1.000 |
| | coefficient | | | | | | | |
| | Sig. (2-tailed) | .045 | .443 | .499 | .026 | .055 | .055 | . |
| | N | 12 | 12 | 12 | 12 | 12 | 12 | 12 |

* The correlation is significant at a level of 0.05 (2-tailed).
** The correlation is significant at a level of 0.01 (2-tailed).

Animal Studies

Ballooned rats (Rats to which the sleeveballoon had been implanted) had a body weight significantly (P<0.0001) lower than sham rats (FIG. 6).

Ballooned animals had blood glucose concentrations after OGTT much lower than sham ones (FIG. 7A). Even circulating insulin levels, both at fasting and after OGTT, were significantly lower (FIG. 7B). The Inventors had found in previous experiments that, when individually infused, both GRP78 and Hsp70 were capable of causing insulin resistance (data not presented).

Continuous infusion of Hsp70 and GRP78 in rats with a standard diet increased glycemia at fasting and that after OGTT, making them similar to the sham ones (FIG. 7A). On the contrary, continuous infusion of monoclonal antibodies against Hsp70 and GRP78 in rats with HFD drastically improved glucose levels (FIG. 7A).

FIG. 7B shows that insulin levels increased when heat shock proteins were infused in rats with a standard diet, and that the infusion of monoclonal antibodies reduced the circulating insulin levels in rats with HFD. Finally, the figure shows that the effects of the sleeveballoon were nullified by the simultaneous infusion of Hsp70 and of GRP78.

Both the HOMA-IR and the AUC of glucose and insulin were significantly lower in sleeveballoon rats than in sham ones (Table 3).

TABLE 3
phosphorylated Akt in skeletal muscle and liver, levels of HOMA-IR, glucose
and insulin AUC and GLP1 (the latter measured at +180 min) after glucose load curve.
Bonferroni inference-corrected significance is reported.

| | | | | | | | Bonferroni |
|---|---|---|---|---|---|---|---|
| | | | | Standard | Standard | | corrected |
| | | | Mean | Deviation | Error | Significance | significance |
| Liver pAkt | 0 | 7 | .5900 | .10786 | .04077 | | |
| | 1 | 7 | .6243 | .15512 | .05863 | 1.000 | |
| | 2 | 7 | .6357 | .16752 | .06332 | 0.999 | |
| | 3 | 7 | 1.7200 | .62671 | .23687 | <0.0001 | <0.001 |
| | 4 | 7 | 1.6671 | .51874 | .19607 | <0.0001 | <0.001 |
| Muscle pAkt | 0 | 7 | .5057 | .14593 | .05516 | | |
| | 1 | 7 | .5200 | .17321 | .06547 | 1.000 | |
| | 2 | 7 | .5486 | .13422 | .05073 | 0.999 | |
| | 3 | 7 | 1.5771 | .34606 | .13080 | <0.0001 | <0.001 |
| | 4 | 7 | 1.3514 | .53502 | .20222 | <0.0001 | <0.001 |
| HOMA_IR | 0 | 7 | 24.0301 | 13.23024 | 5.00056 | | |
| | 1 | 7 | 17.1593 | 8.12517 | 3.07103 | 0.406 | |
| | 2 | 7 | 17.6165 | 2.55167 | .96444 | 0.474 | |
| | 3 | 7 | 7.7608 | 2.78259 | 1.05172 | 0.002 | 0.008 |
| | 4 | 7 | 9.2233 | 2.57018 | .97144 | 0.005 | 0.020 |
| Glucose AUC | 0 | 7 | 72905.7143 | 22528.92722 | 8515.13411 | | |
| | 1 | 7 | 67015.5571 | 22585.54150 | 8536.53229 | 0.953 | |
| | 2 | 7 | 64531.4286 | 10927.52096 | 4130.21470 | 0.851 | |
| | 3 | 7 | 34771.4286 | 7123.35461 | 2692.37497 | 0.001 | 0.004 |
| | 4 | 7 | 38214.2857 | 5441.79767 | 2056.80619 | 0.002 | 0.008 |
| Insulin AUC | 0 | 7 | 701.65257 | 209.237892 | 79.084490 | | |
| | 1 | 7 | 578.60000 | 182.278048 | 68.894626 | 0.513 | |
| | 2 | 7 | 566.63143 | 44.227521 | 16.716432 | 0.421 | |
| | 3 | 7 | 242.74286 | 100.526727 | 37.995531 | <0.0001 | <0.001 |
| | 4 | 7 | 325.27143 | 123.562005 | 46.702048 | <0.0001 | <0.001 |
| GLP1 | 0 | 7 | 3.70 | 0.86 | 0.32 | | |
| | 1 | 7 | 4.21 | 0.61 | 0.23 | 0.310 | |
| | 2 | 7 | 4.11 | 0.79 | 0.30 | 0.176 | |
| | 3 | 7 | 5.53 | 1.31 | 0.50 | 0.028 | 0.112 |
| | 4 | 7 | 5.34 | 1.08 | 0.41 | 0.028 | 0.112 |

Hence, the Inventors found that the infusion of Hsp70 and GRP78 mimics the effects of HFD on glycemia control and nullifies the duodenal bypass effects induced by the sleeveballoon. The infusion of monoclonal antibodies against the two heat shock proteins prevents insulin resistance and diabetes development.

The GLP1 levels at the end of the oral glucose load do not differ significantly, after Bonferroni correction, in all groups compared to the sham-operated ones (Table 3).

In rats with HFD, the sleeveballoon drastically reduces ectopic fat accumulation in the liver, similarly to the infusion of antibodies against Hsp70 and GRP78 (FIG. 8), whereas the infusion of Hsp70 and GRP78 in rats with a normal diet causes NAFLD similarly to sham surgery (data not shown).

Both in muscle (FIG. 9) and in liver (FIG. 10), Akt phosphorylated on Ser 473 was significantly higher in ballooned rats than in sham-operated ones, and in rats receiving anti-Hsp70 and anti-GRP78 antibodies, plus HFD, than in rats infused with Hsp70 and GRP78 and with a standard diet. The effect of the sleeveballoon on Akt was nullified by Hsp70 and GRP78 infusion. Similar data were obtained with GSK phosphorylation (data not shown).

### Study on NASH and Hepatic Fibrosis

Methods

Adult male Wistar rats (n=24) were fed with high-fat, choline-deficient L-amino acid-defined (CDAA) diet that induces NASH and liver fibrosis for 6 weeks (age 90 days at the end of treatment). Rats were continuously infused through a subcutaneous pump with Hsp70/GRP78 monoclonal antibodies (MA) (n=12) or with liquid in which the antibodies are diluted (vehicle control) (n=12), as described above.

At the end of the study, the rats were sacrificed as previously described. Two liver sections (ca. 20×5×2 cm) were taken from the right median lobe and left lateral lobe and fixed for 24 h in formalin, and subsequently embedded in paraffin. The tissue was then sectioned into slices of 5 μm of thickness, mounted on a glass slide and stained by Sirius red (saturated picric acid solution containing 0.1% Direct Red 80 and 0.1% Fast Green FCF) to visualize collagen deposition. Additional tissue sections were instead stained with haematoxylin/eosin. The pathologist was blinded to study design and imaging outcomes (animal groups).

The NAFLD Fibrosis Score (NAS) was calculated according to Kleiner's criteria on haematoxylin/eosin-stained sections (Kleiner D E, Brunt E M, Van Natta M, Behling C, Contos M J, Cummings O W, et al. Design and validation of a histological scoring system for nonalcoholic fatty liver disease. Hepatology. 2005; 41: 1313-1321).

Collagen quantification was performed by a morphometry system [computerized Life Science morphometry system (BIOQUANT, USA)] on a 36 images per animal at 100× magnification (4 picro-sirius red-stained slides per animal, con nine images taken randomly per slide).

Results

At the end of the study, body weight was not statistically different in the two groups (318.17±24.79 vs. 320.67±22.01 g (P=0.796) of rats receiving monoclonal antibodies against Hsp70/GRP78 and in rats infused with vehicle control.

The single components of NAFLD activity score (NAS), which gives a score for the three histological components of NASH, i.e. steatosis, lobular inflammation and ballooning, together with the mean±SD of NAS and statistical significance (Mann-Whitney U test), are reported in Table 4.

TABLE 4

| | Hsp70/GRP78 | |
|---|---|---|
| | Monoclonal antibodies | Vehicle Control |
| Score | Number of rats | Number of rats |
| Steatosis | | |
| 0 | 5 | 0 |
| 1 | 6 | 5 |
| 2 | 1 | 7 |
| 3 | 0 | 0 |
| Lobular inflammation | | |
| 0 | 4 | 0 |
| 1 | 6 | 1 |
| 2 | 2 | 10 |
| 3 | 0 | 1 |
| Hepatocyte ballooning | | |
| 0 | 2 | 0 |
| 1 | 5 | 0 |
| 2 | 4 | 1 |
| 3 | 1 | 11 |
| NAFLD Activity Score | 2.83 ± 1.53 | 5.83 ± 1.11 |
| (NAS) | | |

| | |
|---|---|
| NAS significance | P < 0.0001 |

Scores of steatosis, lobular inflammation, hepatocyte ballooning together with NAFLD Activity Score (NAS), mean ± SD.

The infusion of Hsp70/GRP78 monoclonal antibodies during diet-induced NASH/fibrosis drastically reduces histological NASH features, as well as liver fibrosis.

Administration of Hsp70 and/or GRP78 Recombinant Proteins or their Antibodies

The Inventors observed that by administering HSP70 or GRP78 recombinant proteins in continuous infusion in rats, or by administering their monoclonal antibodies, effects that are lesser than those of a simultaneous administration are had. In other terms, if administered together a potentiation of their effects is observed. However, also single administrations of monoclonal antibodies directed against HSP70 or against GRP78 demonstrate a significant action in insulin resistance reduction.

FIGS. 13 and 14 show glycemia and insulinemia curves after OGTT in rats treated with high-fat diet (HFD) and sham-operated, HFD+Sleeveballoon, HFD+antibody anti-HSP70 and HFD+Sleeveballoon+HSP70 recombinant protein (rHSP70) compared to administration of both antibodies or recombinant proteins.

The HFD+sham-operated and HFD+Sleeveballoon+rHSP70 curves are not significantly different (ANOVA for repeated measures) as well as the HFD+Sleeveballoon and HFD+antibody anti-HSP70 curves. For GRP78, the HFD+sham-operated and HFD+Sleeveballoon+rHSP70 curves are significantly different (P<0.05) in 2 points for glycemia and in 3 points for insulinemia (ANOVA for repeated measures).

Sleeveballoon implantation drastically reduces glycemia and insulinemia despite the high-fat diet, whereas HFD in association with a monoclonal antibody directed against HSP70 or GRP78 improves glycemia and accordingly reduces insulin secretion and insulin resistance. On the contrary, when the HSP70 or GRP78 recombinant protein is infused in animals that, by having the sleeveballoon, had become insulin-sensitive, insulin sensitivity worsens and they become insulin-resistant.

TABLE 5

| | | Glucose AUC | Insulin AUC |
|---|---|---|---|
| | HOMA-IR | (g/dl · min) | (μU/ml · min) |
| HFD +Sham | 20.36 ± 3.58 | 70473.23 ± 17641.47 | 14342.30 ± 6943.04 |
| HFD + | 6.00 ± 1.61 | 37230.03 ± 7907.00 | 4724.20 ± 2604.41 |
| Sleeveballoon | | | |
| HFD + | 16.29 ± 2.63 | 63941.99 ± 8685.76 | 11086.90 ± 1221.8 |
| Sleeveballoon + | | | |
| rHSP70 | | | |
| HFD + | 8.93 ± 0.30 | 39214.3 ± 5804.9 | 6685.01 ± 3031.05 |
| anti-HSP70 | | | |
| antibodies | | | |
| HFD + Sham | 19.31 ± 1.62 | 72905.7 ± 22984.1 | 11720.1 ± 3059.4 |
| HFD + | 6.12 ± 0.17 | 34771.4 ± 7325.3 | 5175.3 ± 648.2 |
| Sleeveballoon | | | |
| HFD + | 15.19 ± 0.64 | 67015.6 ± 23111.8 | 9106.5 ± 5835.1 |
| Sleeveballoon + | | | |
| rGRP78 | | | |
| HFD + | 10.87 ± 3.01 | 42223.48 ± 7916.29 | 8592.6 ± 3236.5 |
| anti-GRP78 | | | |
| antibodies | | | |

insulin resistance (HOMA-IR) and Area Under Curve (AUC) values of glycemia and insulinemia in experiments with HSP70 or GRP78 alone.
HFD + Sleeveballoon vs. HFD + Sham: P < 0.001
HFD + anti-HSP70 antibodies vs. HFD + Sham: P < 0.01
HFD + anti-GRP78 antibodies vs. HFD + Sham: P < 0.05

FIG. 15 shows the glycogen content, highlighted with PAS staining in the skeletal muscle and liver of rats treated with high-fat diet (HFD) and sham-operated, HFD+Sleeveballoon, HFD+anti-HSP70 antibody and HFD+Sleeveballoon+HSP70 recombinant protein (rHSP70).

FIG. 16 shows the lipid content (highlighted with ORO staining) the skeletal muscle and liver of rats treated with high-fat diet (HFD) and sham-operated, HFD+Sleeveballoon, HFD+anti-HSP70 antibody and Sleeveballoon+HSP70 recombinant protein (rHSP70).

FIG. 17 shows the glycogen content highlighted with PAS staining in the skeletal muscle and liver of rats treated with high-fat diet (HFD) and sham-operated, HFD+Sleeveballoon, HFD+anti-GRP78 antibody and HFD+Sleeveballoon+GRP78 recombinant protein (rGRP78).

FIG. 18 shows the lipid content (highlighted with ORO staining) in the skeletal muscle and liver of rats treated with high-fat diet (HFD) and sham-operated, HFD+Sleeveballoon, HFD+anti-GRP78 antibody and Sleeveballoon+GRP78 recombinant protein (rGRP78).

The administration of HSP70 or GRP78, as recombinant proteins or as monoclonal antibodies, has a significant effect on the insulin signal, as seen from FIG. 19. The administration of monoclonal antibodies against HSP70 or GRP78 in the presence of HFD has significantly increased Akt phosphorylation on the serine residue in position 473, whereas the administration of recombinant protein in rats with Sleeveballoon has significantly reduced phosphorylation, but with significance values higher in the case of HSP70 (P<0.01) than in that of GRP78 (P<0.05).

Jejunal Biopsy

Jejunal biopsies were obtained during enteroscopy in 7 subjects with histological proven NASH, obesity and insulin resistance and in 7 healthy volunteers.

Insulin Sensitivity Assessment

Insulin sensitivity was assessed by the euglycemic hyperinsulinemic clamp (DeFronzo R A, Tobin J D, Andres R (1979) Glucose clamp technique: a method for quantifying insulin secretion and resistance. Am J Physiol 237: E214-E223). Insulin sensitivity was determined during the last 40 min of the clamp by computing the glucose amount infused (M, μmol·min⁻¹·kg_{bw}⁻¹) needed to maintain euglycemia, i.e. glycemia in a normal range measured at fasting.

Intestinal Protein Secretion

Jejunal mucosa biopsies were washed with saline and half used to measure intracellular proteins and half incubated in oxygenated (O₂:CO₂, 95:5, v/v) Krebs-Henseleit solution (37° C., pH 7.4) in vitro for 1 h to isolate proteins secreted into the medium (secretoma). Conditioned medium was lyophilized and stored at −80° C. for subsequent protein purification.

Proteomics

Proteins were digested by trypsin, labelled with TMT8plex including an internal reference, followed by SCX fractionation and LC-MS/MS (MS3) analysis (EASY-nLC II NSI Orbitrap Velos (Thermo) system). Proteome Discoverer 1.3 (Thermo) software was used for data processing and protein identification.

DI: Differential intensity ratio, defined as the exponentiated difference of intensity averages, between the group of interest (group A) and the base group (group B).

Colors indicate the direction and amplitude of changes.

An increase of a group A (patients with NASH, insulin resistance and obesity) protein compared to controls is expressed in various shades of red, whereas a reduction of the levels of a given protein is expressed in shades of blue.

p-Value: statistical significance (p≤1.05) of the peptide level

q-Value: adjusted p-Value to correct for multiple testing using Storey's method (grey shading: p≤1.05)

DR_A: detection rate (DR) in group A, defined as the proportion of samples with an intensity greater than or equal to the Intensity Threshold.

DR_B: detection rate (DR) in group B, defined as the proportion of samples with an intensity greater than or equal to the Intensity Threshold.

Results

Patients with NASH had a significantly higher weight and BMI and a significantly lower insulin mediated glucose uptake than healthy controls, but were matched by height, age and sex. The average NAFLD Fibrosis Score (NAS) was 4.43±1.27, indicating the histological presence of NASH (Table 6).

Proteomics data are reported as the ratio between patients/controls, therefore a value of >1 indicates an increase of a certain protein expression in patients as compared with controls, and vice versa. GRP74 and GRP78, as well as the members of the HSP70 family were all increased in the conditioned medium of subjects with obesity, insulin resistance and NASH as compared with healthy controls.

TABLE 6
anthropometric data, glucose amount infused during clamp and NAS.

| | | Weight | BMI | | | | |
|---|---|---|---|---|---|---|---|
| | Height (cm) | (kg) | (kg/m\hat{ }2) | age (yrs) | sex (0F, 1M) | M (mg/kg/mm) | NAS |
| PATIENTS | | | | | | | |
| 1 | 161 | 98 | 37.81 | 45 | 0 | 4.12 | 3 |
| 2 | 185 | 135 | 39.44 | 36 | 1 | 2.79 | 5 |
| 3 | 156 | 102 | 41.91 | 52 | 0 | 3.15 | 4 |
| 4 | 168 | 146 | 51.73 | 61 | 0 | 1.56 | 6 |
| 5 | 180 | 127 | 39.20 | 45 | 1 | 3.27 | 4 |
| 6 | 164 | 110 | 40.90 | 57 | 0 | 4.11 | 3 |
| 7 | 179 | 148 | 46.19 | 49 | 1 | 1.39 | 6 |
| | 170.43 | 123.71 | 42.45 | 49.29 | 0.43 | 2.91 | 4.43 |
| | 10.97 | 20.60 | 4.90 | 8.34 | 0.53 | 1.10 | 1.27 |
| CONTROLS | | | | | | | |
| 1 | 185 | 85 | 24.84 | 47 | 1 | 6.54 | 0 |
| 2 | 192 | 92 | 24.96 | 53 | 1 | 6.26 | 0 |
| 3 | 164 | 55 | 20.45 | 62 | 0 | 9.15 | 1 |
| 4 | 158 | 57 | 22.83 | 35 | 0 | 8.31 | 0 |
| 5 | 169 | 61 | 21.36 | 41 | 0 | 8.55 | 1 |
| 6 | 170 | 70 | 24.22 | 55 | 0 | 7.15 | 0 |
| 7 | 183 | 83 | 24.78 | 53 | 1 | 6.96 | 0 |
| | 174.43 | 71.86 | 23.35 | 49.43 | 0.43 | 7.56 | 0.29 |
| | 12.39 | 14.88 | 1.84 | 9.13 | 0.53 | 1.11 | 0.49 |
| Significance | 0.535 | 0.000 | 0.000 | 0.976 | 1.000 | 0.000 | 0.000 |

Table 7, which shows the differential intensity ratio, demonstrates that the levels of GRP78, but also of GRP74, deriving from deletion of the C-terminal KDEL motif, are higher in the gut mucosa secretome in subjects with obesity, insulin resistance and NASH as compared with healthy volunteers. It is known that the lack of the C-terminal KDEL motif allows GRP78 secretion. This can explain the high levels GRP78 in the circulatory stream after a high-fat, high-sucrose diet in humans.

SRP75, HSP71, HSP74 and HSP76 belong, as mentioned above, to the HSP70 family.

Jejunal mucosa secretoma

| UNIPROT_ID | ISOTOPE_GROUP_ID | DI | p-Value | q-Value | DR_A | DR_B |
|---|---|---|---|---|---|---|
| GRP75_HUMAN | 1_151285325 | 1.18 | 0 | 0 | 100% | 100% |
| GRP75_HUMAN | 1_151286460 | 1.09 | 0.007 | 0.005 | 100% | 100% |
| GRP75_HUMAN | 1_151286659 | 1.23 | 0.006 | 0.005 | 100% | 100% |
| GRP75_HUMAN | 1_151288525 | 1 | 0.996 | 0.229 | 100% | 100% |
| GRP75_HUMAN | 1_151289564 | 1.05 | 0.074 | 0.03 | 100% | 100% |
| GRP75_HUMAN | 1_151290255 | 0.93 | 0.009 | 0.006 | 100% | 100% |
| GRP75_HUMAN | 1_151290768 | 1.06 | 0.222 | 0.069 | 100% | 100% |
| GRP75_HUMAN | 1_151291634 | 1.01 | 0.971 | 0.224 | 100% | 100% |
| GRP75_HUMAN | 1_151292646 | 1.08 | 0.095 | 0.037 | 100% | 100% |
| GRP75_HUMAN | 1_151292971 | 0.98 | 0.472 | 0.126 | 100% | 100% |
| GRP75_HUMAN | 1_151293108 | 1.02 | 0.446 | 0.121 | 100% | 100% |
| GRP75_HUMAN | 1_151293875 | 1.02 | 0.545 | 0.142 | 100% | 100% |
| GRP75_HUMAN | 1_151295229 | 1.05 | 0.419 | 0.115 | 100% | 100% |
| GRP75_HUMAN | 1_151297111 | 1.01 | 0.769 | 0.187 | 100% | 100% |
| GRP75_HUMAN | 1_151298313 | 0.94 | 0.054 | 0.024 | 100% | 100% |
| GRP75_HUMAN | 1_151299831 | 0.93 | 0.001 | 0.001 | 100% | 100% |
| GRP75_HUMAN | 1_151300138 | 1.16 | 0.003 | 0.003 | 100% | 100% |
| GRP75_HUMAN | 1_151300866 | 1.14 | 0 | 0 | 100% | 100% |
| GRP75_HUMAN | 1_151301374 | 1.56 | 0.44 | 0.119 | 100% | 80% |
| GRP75_HUMAN | 1_151302086 | 1.04 | 0.252 | 0.077 | 100% | 100% |
| GRP75_HUMAN | 1_151303210 | 0.93 | 0.132 | 0.047 | 100% | 100% |
| GRP75_HUMAN | 1_151306012 | 0.87 | 0 | 0.001 | 100% | 100% |
| GRP75_HUMAN | 1_151307335 | 0.83 | 0.002 | 0.002 | 100% | 100% |
| GRP75_HUMAN | 1_151312351 | 1.08 | 0.102 | 0.039 | 100% | 100% |
| GRP75_HUMAN | 1_151312977 | 0.97 | 0.116 | 0.042 | 100% | 100% |
| GRP75_HUMAN | 1_151317325 | 0.85 | 0.005 | 0.004 | 100% | 100% |
| GRP75_HUMAN | 1_151319290 | 1.16 | 0.013 | 0.008 | 100% | 100% |
| GRP75_HUMAN | 1_151319609 | 1.07 | 0.002 | 0.002 | 100% | 100% |
| GRP75_HUMAN | 1_151321822 | 0.9 | 0.004 | 0.004 | 100% | 100% |
| GRP75_HUMAN | 1_151323647 | 1.04 | 0.252 | 0.077 | 100% | 100% |
| GRP75_HUMAN | 1_151323761 | 1.04 | 0.199 | 0.064 | 100% | 100% |
| GRP75_HUMAN | 1_151325995 | 1.14 | 0.006 | 0.005 | 100% | 100% |
| GRP75_HUMAN | 1_151328225 | 0.9 | 0 | 0 | 100% | 100% |
| GRP75_HUMAN | 1_151330125 | 1.14 | 0 | 0 | 100% | 100% |
| GRP75_HUMAN | 1_151331925 | 1.11 | 0.003 | 0.003 | 100% | 100% |
| GRP75_HUMAN | 1_151332045 | 1.58 | 0.367 | 0.103 | 100% | 80% |
| GRP75_HUMAN | 1_151334817 | 0.88 | 0.003 | 0.003 | 100% | 100% |
| GRP75_HUMAN | 1_151335106 | 1.06 | 0.141 | 0.049 | 100% | 100% |
| GRP75_HUMAN | 1_151336465 | 0.98 | 0.715 | 0.177 | 100% | 100% |
| GRP75_HUMAN | 1_151338989 | 1.56 | 0.403 | 0.111 | 100% | 80% |
| GRP75_HUMAN | 1_151342397 | 2.48 | 0.369 | 0.104 | 80% | 40% |
| GRP75_HUMAN | 1_151342846 | 1.94 | 0.106 | 0.04 | 100% | 80% |
| GRP75_HUMAN | 1_151350305 | 1 | 0.943 | 0.219 | 100% | 100% |
| GRP75_HUMAN | 1_151361253 | 0.98 | 0.711 | 0.176 | 100% | 100% |
| GRP75_HUMAN | 1_151376139 | 0.55 | 0.231 | 0.072 | 80% | 100% |
| GRP75_HUMAN | 1_151384132 | 0.92 | 0.004 | 0.004 | 100% | 100% |
| GRP75_HUMAN | 1_151391976 | 1.06 | 0.16 | 0.054 | 100% | 100% |
| GRP75_HUMAN | 1_151412802 | 0.76 | 0.695 | 0.173 | 80% | 80% |
| GRP75_HUMAN | 1_151467020 | 0.56 | 0.475 | 0.127 | 80% | 100% |
| GRP75_HUMAN \| | 1_151305867 | 1.02 | 0.729 | 0.179 | 100% | 100% |
| HS71L_HUMAN \| | | | | | | |
| HSP71_HUMAN | | | | | | |
| GRP78_HUMAN | 1_151279033 | 0.95 | 0.005 | 0.004 | 100% | 100% |
| GRP78_HUMAN | 1_151279282 | 0.99 | 0.737 | 0.181 | 100% | 100% |
| GRP78_HUMAN | 1_151279311 | 0.88 | 0 | 0 | 100% | 100% |
| GRP78_HUMAN | 1_151279342 | 1.1 | 0.034 | 0.017 | 100% | 100% |
| GRP78_HUMAN | 1_151279774 | 0.92 | 0 | 0 | 100% | 100% |
| GRP78_HUMAN | 1_151279839 | 0.85 | 0.001 | 0.002 | 100% | 100% |
| GRP78_HUMAN | 1_151279976 | 0.86 | 0 | 0 | 100% | 100% |
| GRP78_HUMAN | 1_151280260 | 1 | 0.891 | 0.21 | 100% | 100% |
| GRP78_HUMAN | 1_151280428 | 1.13 | 0 | 0 | 100% | 100% |
| GRP78_HUMAN | 1_151280990 | 1.2 | 0.003 | 0.003 | 100% | 100% |
| GRP78_HUMAN | 1_151281794 | 1.09 | 0.05 | 0.022 | 100% | 100% |
| GRP78_HUMAN | 1_151281797 | 0.92 | 0.001 | 0.002 | 100% | 100% |
| GRP78_HUMAN | 1_151281832 | 0.85 | 0.05 | 0.023 | 100% | 100% |
| GRP78_HUMAN | 1_151281859 | 0.91 | 0.003 | 0.003 | 100% | 100% |
| GRP78_HUMAN | 1_151281875 | 0.98 | 0.493 | 0.131 | 100% | 100% |
| GRP78_HUMAN | 1_151282421 | 0.97 | 0.46 | 0.124 | 100% | 100% |
| GRP78_HUMAN | 1_151282980 | 1.07 | 0.12 | 0.044 | 100% | 100% |
| GRP78_HUMAN | 1_151282986 | 1.09 | 0.025 | 0.014 | 100% | 100% |
| GRP78_HUMAN | 1_151283009 | 1.11 | 0.048 | 0.022 | 100% | 100% |
| GRP78_HUMAN | 1_151283688 | 0.89 | 0.002 | 0.002 | 100% | 100% |
| GRP78_HUMAN | 1_151284258 | 0.94 | 0.133 | 0.047 | 100% | 100% |
| GRP78_HUMAN | 1_151284415 | 1.03 | 0.305 | 0.089 | 100% | 100% |
| GRP78_HUMAN | 1_151284428 | 1.08 | 0.011 | 0.007 | 100% | 100% |
| GRP78_HUMAN | 1_151284566 | 0.85 | 0 | 0.001 | 100% | 100% |
| GRP78_HUMAN | 1_151285336 | 1.04 | 0.338 | 0.097 | 100% | 100% |
| GRP78_HUMAN | 1_151286034 | 0.82 | 0 | 0.001 | 100% | 100% |
| GRP78_HUMAN | 1_151286131 | 1.07 | 0.029 | 0.015 | 100% | 100% |
| GRP78_HUMAN | 1_151286306 | 1.12 | 0 | 0 | 100% | 100% |
| GRP78_HUMAN | 1_151286905 | 1.1 | 0.04 | 0.019 | 100% | 100% |
| GRP78_HUMAN | 1_151287722 | 1.14 | 0.001 | 0.001 | 100% | 100% |
| GRP78_HUMAN | 1_151287921 | 1.07 | 0.014 | 0.009 | 100% | 100% |
| GRP78_HUMAN | 1_151288811 | 0.9 | 0.019 | 0.011 | 100% | 100% |
| GRP78_HUMAN | 1_151289310 | 0.92 | 0.069 | 0.029 | 100% | 100% |
| GRP78_HUMAN | 1_151289555 | 0.85 | 0.001 | 0.001 | 100% | 100% |
| GRP78_HUMAN | 1_151289571 | 1 | 0.991 | 0.228 | 100% | 100% |
| GRP78_HUMAN | 1_151289687 | 1.05 | 0.078 | 0.032 | 100% | 100% |
| GRP78_HUMAN | 1_151290129 | 0.89 | 0.001 | 0.001 | 100% | 100% |
| GRP78_HUMAN | 1_151290214 | 0.91 | 0.074 | 0.03 | 100% | 100% |
| GRP78_HUMAN | 1_151293867 | 1.11 | 0.021 | 0.012 | 100% | 100% |
| GRP78_HUMAN | 1_151293893 | 1.03 | 0.122 | 0.044 | 100% | 100% |
| GRP78_HUMAN | 1_151295908 | 0.48 | 0.032 | 0.016 | 80% | 100% |
| GRP78_HUMAN | 1_151298346 | 1.06 | 0.466 | 0.125 | 100% | 100% |
| GRP78_HUMAN | 1_151298698 | 0.93 | 0.014 | 0.009 | 100% | 100% |
| GRP78_HUMAN | 1_151300075 | 1.09 | 0.04 | 0.019 | 100% | 100% |
| GRP78_HUMAN | 1_151301112 | 1.05 | 0.481 | 0.128 | 100% | 100% |
| GRP78_HUMAN | 1_151301216 | 1.08 | 0.069 | 0.029 | 100% | 100% |
| GRP78_HUMAN | 1_151301503 | 1.26 | 0 | 0.001 | 100% | 100% |
| GRP78_HUMAN | 1_151304761 | 0.87 | 0.009 | 0.006 | 100% | 100% |
| GRP78_HUMAN | 1_151307624 | 0.97 | 0.189 | 0.061 | 100% | 100% |
| GRP78_HUMAN | 1_151312826 | 0.77 | 0.014 | 0.009 | 100% | 100% |
| GRP78_HUMAN | 1_151312863 | 1.26 | 0 | 0 | 100% | 100% |
| GRP78_HUMAN | 1_151313164 | 1.13 | 0.007 | 0.005 | 100% | 100% |
| GRP78_HUMAN | 1_151313440 | 1.09 | 0.009 | 0.006 | 100% | 100% |
| GRP78_HUMAN | 1_151315499 | 0.92 | 0.03 | 0.015 | 100% | 100% |
| GRP78_HUMAN | 1_151318943 | 1.07 | 0.079 | 0.032 | 100% | 100% |
| GRP78_HUMAN | 1_151320855 | 0.93 | 0.055 | 0.024 | 100% | 100% |
| GRP78_HUMAN | 1_151323025 | 1.02 | 0.453 | 0.122 | 100% | 100% |
| GRP78_HUMAN | 1_151323115 | 1.08 | 0.182 | 0.06 | 100% | 100% |
| GRP78_HUMAN | 1_151326899 | 0.91 | 0.023 | 0.012 | 100% | 100% |
| GRP78_HUMAN | 1_151330851 | 2.01 | 0.089 | 0.035 | 100% | 80% |
| GRP78_HUMAN | 1_151334552 | 1.04 | 0.125 | 0.045 | 100% | 100% |
| GRP78_HUMAN | 1_151339699 | 0.52 | 0.083 | 0.033 | 80% | 100% |
| GRP78_HUMAN | 1_151340284 | 0.26 | 0.026 | 0.014 | 60% | 100% |
| GRP78_HUMAN | 1_151340734 | 1.01 | 0.689 | 0.171 | 100% | 100% |
| GRP78_HUMAN | 1_151341209 | 0.91 | 0.029 | 0.015 | 100% | 100% |
| GRP78_HUMAN | 1_151344985 | 1.65 | 0.267 | 0.08 | 100% | 80% |
| GRP78_HUMAN | 1_151356082 | 2.66 | 0.259 | 0.078 | 100% | 60% |
| GRP78_HUMAN | 1_151360302 | 1.75 | 0.172 | 0.057 | 100% | 80% |
| GRP78_HUMAN | 1_151361192 | 1.15 | 0.022 | 0.012 | 100% | 100% |
| GRP78_HUMAN | 1_151361280 | 1.05 | 0.02 | 0.012 | 100% | 100% |
| GRP78_HUMAN | 1_151362224 | 1.04 | 0.228 | 0.071 | 100% | 100% |
| GRP78_HUMAN | 1_151365138 | 1.16 | 0.004 | 0.003 | 100% | 100% |
| GRP78_HUMAN | 1_151372391 | 1.11 | 0.042 | 0.02 | 100% | 100% |
| GRP78_HUMAN | 1_151372470 | 1.06 | 0.266 | 0.08 | 100% | 100% |
| GRP78_HUMAN | 1_151380303 | 1.05 | 0.083 | 0.033 | 100% | 100% |
| GRP78_HUMAN | 1_151380448 | 2.21 | 0.031 | 0.016 | 100% | 80% |
| GRP78_HUMAN | 1_151385580 | <0.1 | 0 | 0 | 0% | 100% |
| GRP78_HUMAN | 1_151387395 | 0.94 | 0.021 | 0.012 | 100% | 100% |
| GRP78_HUMAN | 1_151389638 | 0.93 | 0.016 | 0.01 | 100% | 100% |
| GRP78_HUMAN | 1_151396266 | 2.53 | 0.192 | 0.062 | 80% | 40% |
| GRP78_HUMAN | 1_151425935 | 0.57 | 0.179 | 0.059 | 80% | 100% |
| GRP78_HUMAN | 1_151434071 | 0.8 | 0 | 0.001 | 100% | 100% |
| HSP71_HUMAN | 1_151284471 | 0.98 | 0.663 | 0.166 | 100% | 100% |
| HSP71_HUMAN | 1_151286296 | 0.96 | 0.018 | 0.011 | 100% | 100% |
| HSP71_HUMAN | 1_151289932 | 0.9 | 0.12 | 0.044 | 100% | 100% |
| HSP71_HUMAN | 1_151291763 | 1.08 | 0.003 | 0.003 | 100% | 100% |
| HSP71_HUMAN | 1_151291945 | 0.99 | 0.908 | 0.213 | 100% | 100% |
| HSP71_HUMAN | 1_151293865 | 0.99 | 0.747 | 0.183 | 100% | 100% |
| HSP71_HUMAN | 1_151302732 | 0.95 | 0.153 | 0.052 | 100% | 100% |
| HSP71_HUMAN | 1_151339138 | 0.89 | 0.024 | 0.013 | 100% | 100% |
| HSP71_HUMAN | 1_151345825 | 0.95 | 0.401 | 0.111 | 100% | 100% |
| HSP74_HUMAN | 1_151284344 | 0.82 | 0 | 0 | 100% | 100% |
| HSP74_HUMAN | 1_151298632 | 1.2 | 0.001 | 0.001 | 100% | 100% |
| HSP74_HUMAN | 1_151322098 | 1.72 | 0.273 | 0.082 | 100% | 80% |
| HSP74_HUMAN | 1_151323221 | 1 | 0.98 | 0.226 | 100% | 100% |
| HSP74_HUMAN | 1_151327948 | 1.75 | 0.149 | 0.051 | 100% | 80% |
| HSP74_HUMAN | 1_151352921 | 0.94 | 0.242 | 0.074 | 100% | 100% |
| HSP74_HUMAN | 1_151353355 | 1 | 0.933 | 0.218 | 100% | 100% |
| HSP74_HUMAN | 1_151354440 | 0.77 | 0 | 0 | 100% | 100% |
| HSP76_HUMAN | 1_151299336 | 1.28 | 0.335 | 0.096 | 100% | 100% |
| HSP76_HUMAN \| | 1_151284874 | 0.98 | 0.129 | 0.046 | 100% | 100% |
| HSP71_HUMAN | | | | | | |
| HSP76_HUMAN \| | 1_151287557 | 1.16 | 0.005 | 0.004 | 100% | 100% |
| HSP71_HUMAN | | | | | | |
| HSP76_HUMAN \| | 1_151297059 | 1.15 | 0 | 0 | 100% | 100% |
| HSP71_HUMAN | | | | | | |
| HSP76_HUMAN \| | 1_151314253 | 1.08 | 0.101 | 0.038 | 100% | 100% |
| HSP71_HUMAN | | | | | | |
| HSP76_HUMAN \| | 1_151329000 | 0.94 | 0.215 | 0.068 | 100% | 100% |
| HSP71_HUMAN | | | | | | |
| HSP76_HUMAN \| | 1_151329228 | 5.42 | 0.008 | 0.006 | 100% | 40% |
| HSP71_HUMAN | | | | | | |
| HSP76_HUMAN \| | 1_151362121 | 1.21 | 0.002 | 0.002 | 100% | 100% |
| HSP71_HUMAN | | | | | | |
| HSP76_HUMAN \| | 1_151281376 | 0.89 | 0.002 | 0.002 | 100% | 100% |
| HSP7C_HUMAN \| | | | | | | |
| HSP72_HUMAN | | | | | | |
| HSP76_HUMAN \| | 1_151282279 | 0.88 | 0 | 0.001 | 100% | 100% |
| HSP7C_HUMAN \| | | | | | | |
| HSP72_HUMAN | | | | | | |

Members of the HSP70 family:
Uniprot_ID: GRP75_HUMAN; Protein description: Stress-70 protein, mitochondrial precursor
Uniprot_ID: GRP78_HUMAN; Protein description: 78 kDa glucose-regulated protein precursor
Uniprot_ID: HSP71_HUMAN; Protein description: Heat shock 70 kDa protein 1A/1B
Uniprot_ID: HSP76_HUMAN; Protein description: Heat shock 70 kDa protein 6; https://www.uniprot.org/
Uniprot ID: HSP72 (synonym of HSP70)

DI: Differential intensity ratio, defined as the exponentiated difference of intensity averages, between the group of interest, subjects with NASH, insulin resistance and obesity (Group A) and the control group of healthy volunteers (Group B). The color shading of DI values is a function of the direction and amplitude of the percentage variations observed. An increase in group A is indicated in shades of red, whereas a reduction is indicated in shades of blue.

p-Value: Statistical significance (P<0.05) of the peptide level.

q-Value: adjusted P-value to correct for multiple testing using Storey's method (P<0.05).

DR_A: Detection rate (DR) in Group A, defined as the proportion of samples with an intensity greater than or equal to the intensity threshold.

DR_B: Detection rate (DR) in Group B, defined as the proportion of samples with an intensity greater than or equal to the intensity threshold.

1. A method of treating insulin resistance and/or pathologies deriving therefrom or related thereto comprising administering to a subject in need thereof an inhibitor of the activity of at least one protein belonging to the Hsp70 family and/or inhibitors of the GRP78 protein activity, wherein said inhibitors of the Hsp70 activity are selected from one or more of anti-Hsp70 monoclonal antibodies or their antigen-binding fragments, molecules of interfering RNAs that bind uniquely to mRNA encoding Hsp70 or a protein belonging to the HSP70 family, and wherein said inhibitors of the GRP78 activity are selected from one or more of anti-GRP78 monoclonal antibodies or their antigen-binding fragments, or molecules of interfering RNAs that bind uniquely to mRNA encoding GRP78.

2. The method according to claim 1, wherein said proteins belonging to the HSP70 family are HSP70, HSP71, HSP76, HSP74, GRP75.

3. The method according to claim 1, wherein said molecules of interfering RNAs are siRNAs specific for RNA encoding Hsp70 or a protein belonging to the HSP70 family and/or siRNAs specific for RNA encoding GRP78.

4. The method according to claim 3, wherein said siRNAs are selected from SEQ ID NOs: 1 to 11.

5. The method according to claim 1, wherein said molecules of interfering RNAs are formulated so as to be released in the intestine.

6. The method according to claim 1, wherein said pathologies deriving from insulin resistance or related to insulin resistance are hyperglycemia, type 2 diabetes, metabolic syndrome, atherosclerosis, polycystic ovary syndrome, dyslipidemia, obesity, cardiovascular disease, hypertension, NASH, NAFLD and hepatic fibrosis.

7. The method according to claim 1, wherein said inhibitors are administered simultaneously or sequentially to a patient in need thereof.

8. A method of treating insulin resistance and/or pathologies deriving therefrom or related thereto comprising administering to a patient in need thereof a composition comprising an inhibitor of the activity of at least one protein belonging to the Hsp70 family and/or inhibitors of the GRP78 protein activity and one or more excipients or pharmaceutically acceptable carriers, wherein said inhibitors of the Hsp70 activity are selected from one or more of anti-Hsp70 monoclonal antibodies or their antigen-binding fragments, molecules of interfering RNAs that bind uniquely to mRNA encoding Hsp70 or a protein belonging to the HSP70 family, and wherein said inhibitors of the GRP78 activity are selected from one or more of anti-GRP78 monoclonal antibodies or their antigen-binding fragments, or molecules of interfering RNAs that bind uniquely to mRNA encoding GRP78.

9. The method according to claim 8, wherein said proteins belonging to the HSP70 family are HSP70, HSP71, HSP76, HSP74, GRP75.

10. The method according to claim 9, wherein said molecules of interfering RNAs are siRNAs specific for RNA encoding Hsp70 or for a protein belonging to the HSP70 family and/or siRNAs specific for RNA encoding GRP78.

11. The method according to claim 8, wherein said siRNAs are selected from one or more of SEQ ID NOs: 1 to 12.

12. The method according to claim 8, wherein said pathologies deriving from insulin resistance—or related to insulin resistance are hyperglycemia, type 2 diabetes, metabolic syndrome, atherosclerosis, polycystic ovary syndrome, dyslipidemia, obesity, cardiovascular disease, hypertension, NASH, NAFLD and hepatic fibrosis.

13. The method according to claim 8, wherein said inhibitors are co-formulated or are provided in separate aliquots for simultaneous or sequential administration.

14. The method according to claim 8, in formulation for oral, systemic, intranasal, or intravenous administration.

15. The method according to claim 8, wherein said molecules of interfering RNAs are formulated so as to be released in the intestine.

16. The method according to claim 15, wherein the composition is for gastro-resistant oral use.

17. The method according to claim 16, wherein said composition comprises lipid nanoparticles loaded with said molecules of interfering RNAs.

18. The method according to claim 8, wherein said composition comprises one or more anti-GRP78 monoclonal antibodies or their antigen-binding fragments and/or one or more anti-Hsp70 monoclonal antibodies or their antigen-binding fragments for Hsp70 protein or one or more monoclonal antibodies or antigen-binding fragments thereof that specifically bind a protein belonging to the HSP70 family, and wherein said composition is formulated for subcutaneous administration.

19. The method according to claim 8, wherein said inhibitors are administered simultaneously or sequentially to a patient in need thereof.

20. The method according to claim 19, wherein the antibodies are administered subcutaneously and said molecules of interfering RNAs are administered orally in gastro-resistant formulations.

21. A method for diagnosing insulin resistance in a human comprising:
a. measuring the concentration of a blood sample of Hsp70 and GRP78 proteins, and wherein
b. an overall blood concentration of said proteins higher than 256 pg/ml is indicative of insulin resistance.
